(19) United States
(12) Patent Application Publication
(10) Pub. No.: US 2022/0215906 A1
(43) Pub. Date: Jul. 7, 2022

(54) CANCER MEDICAL DRUG TREATMENTS ASSAY METHOD AND DEVICES

(71) Applicants: Cary A. Presant, San Marino, CA (US); Russell Garry Latimer, Brentwood, TN (US); Willard Watson Young, III, Brentwood, TN (US)

(72) Inventors: Cary A. Presant, San Marino, CA (US); Russell Garry Latimer, Brentwood, TN (US); Willard Watson Young, III, Brentwood, TN (US)

(21) Appl. No.: 17/692,493

(22) Filed: Mar. 11, 2022

Related U.S. Application Data
(63) Continuation of application No. 16/435,445, filed on Jun. 7, 2019.

(51) Int. Cl. G16C 20/30; G16H 50/20

(52) U.S. Cl. CPC G16C 20/30; G16H 50/20

(57) ABSTRACT

The embodiments disclose a method including interpreting of APOP results for a series of drugs or combinations and creating a direct APOP assay of purified cells, creating suggested clinician decisions based on the direct APOP assay of purified cells results in choosing potential treatments each when combined with genomic changes identified by next generation testing of tumor DNA from purified cells, identifying nonequivalence of drugs in the APOP assay or other tests, identifying cannabinoid/CBD anti-tumor effects or immune-activity effects or enhancement of other drug anti-tumor effects in the APOP assay or other tests, and using a direct APOP assay of purified cells application for transmitting direct APOP assay data.

### BACKGROUND

The health care industry is facing difficulties with spiraling higher costs. Clinicians face having to make drug treatment choices for patients from a myriad of new drugs designed to combat a myriad of conditions and diseases. Drug treatment choices may have been through trials that do not match a particular patient's genetic response or stage of condition. A clinician needs to be aware of the clinical and cost effectiveness of all drugs or combinations of drugs before using those treatments on a patient.

### BRIEF DESCRIPTION OF THE DRAWINGS

FIG. 1 shows for illustrative purposes only an example of an overview of a method and devices for direct apoptosis assay of purified cells of one embodiment.

FIG. 2 shows a block diagram of an overview of a cancer companion diagnostic for chemotherapy of one embodiment.

FIG. 3 shows a block diagram of an overview flow chart of performing a cancer companion diagnostic direct apoptosis assay of purified cancer cells of one embodiment.

FIG. 4 shows a block diagram of an overview flow chart of receiving patient biopsy tissue sample of one embodiment.

FIG. 5 shows a block diagram of an overview flow chart of assaying apoptosis of purified cancer cells in culture of one embodiment.

FIG. 6 shows a block diagram of an overview flow chart of a direct APOP assay of purified cells of one embodiment.

FIG. 7 shows a block diagram of an overview of 1.0 direct APOP assay of purified cells of one embodiment.

FIG. 8 shows a block diagram of an overview of 1.05 purified cells+drugs of one embodiment.

FIG. 9 shows a block diagram of an overview of 1.1 culture and determination of antitumor activity of one embodiment.

FIG. 10 shows a block diagram of an overview of 2.0 using APOP for anti-inflammatory therapy of one embodiment.

FIG. 11 shows a block diagram of an overview of 3.0 using APOP for anti-immunological therapy of one embodiment.

FIG. 12 shows a block diagram of an overview of 4.0 using APOP to increase immune therapy effects of one embodiment.

FIG. 13 shows a block diagram of an overview of 5.0 extended APOP assay decision tree of one embodiment.

FIG. 14 shows a block diagram of an overview of 6.0 pre-APOP assay decision tree of one embodiment.

FIG. 15 shows a block diagram of an overview of 7.0 parallel APOP assay decision tree of one embodiment.

FIG. 16 shows a block diagram of an overview of 8.0 interpretations of APOP results for a series of drugs or combinations of one embodiment.

FIG. 17A shows a block diagram of an overview of 9.0 using the APOP assay on therapy of patients with resistant or heavily pretreated cancer of one embodiment.

FIG. 17B shows a block diagram of an overview of situations of one embodiment.

FIG. 17C shows a block diagram of an overview of situations continued of one embodiment.

FIG. 18 shows a block diagram of an overview of 10.0 interpretations of APOP results for drugs or combinations based on amount of O.D. change of one embodiment.

FIG. 19 shows a block diagram of an overview of 11.0 interpretations of APOP results for drugs with similar mechanisms of action of one embodiment.

FIG. 20 shows a block diagram of an overview of 12.0 advanced interpretation of APOP results using O.D. change and maximum O.D. increase from a single drug or combination of one embodiment.

FIG. 21 shows a block diagram of an overview of 13.0 enhancing drug development decisions by use of APOP assay and cell growth inhibition of one embodiment.

FIG. 22 shows a block diagram of an overview of 14.0 a method to reduce cost of chemotherapy and/or drug therapy for cancer of one embodiment.

FIG. 23 shows a block diagram of an overview of cost of drugs or therapies defined of one embodiment.

FIG. 24A shows a block diagram of an overview of 15.0 a method to promote immune therapy effects of immuno-active drugs and/or immune cells in treating cancer or leukemia of one embodiment.

FIG. 24B shows a block diagram of an overview of 15.1 cancer or leukemia cells of one embodiment.

FIG. 24C shows a block diagram of an overview of 16.0 a method to evaluate whether to consider using immunoactive drugs to treat cancer of one embodiment.

FIG. 25 shows a block diagram of an overview of measure immune marker before APOP assay of one embodiment.

FIG. 26 shows a block diagram of an overview of 15.2 APOP assay cancer cells of one embodiment.

FIG. 27A shows a block diagram of an overview of 17.0 a method to identify non-equivalences of drugs of one embodiment.

FIG. 27B shows a block diagram of an overview of 17.1 using the APOP assay of one embodiment.

FIG. 28 shows a block diagram of an overview of 18.0 a method for identifying an anti-apoptosis drug of one embodiment.

FIG. 29 shows for illustrative purposes only an example of direct APOP assay of purified cells application of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

### General Overview:

It should be noted that the descriptions that follow, for example, in terms of a method and devices for direct APOP assay of purified cells is described for illustrative purposes and the underlying system can apply to any number and multiple types of medical drug treatments. In one embodiment of the present invention, the method and devices for direct APOP assay of purified cells can be configured using a number of drugs for testing. The method and devices for direct APOP assay of purified cells may be configured to include a number of cell purification technologies and may be configured to include a number of next-generation sequencing technologies using the present invention.

The term “apoptosis” used herein refers to a genetically directed process of cell self-destruction that is marked by the fragmentation of nuclear DNA, is activated either by the presence of a stimulus or removal of a suppressing agent or stimulus, is a normal physiological process eliminating DNA-damaged, superfluous, or unwanted cells, and when halted (as by genetic mutation) may result in uncontrolled cell growth and tumor formation and additionally is expressed without any change in meaning as “APOP” in any case lower, upper or mixed.

The term “APOP” used herein refers to an assay to test and measure apoptosis effectiveness of a single drug or combination of drugs against purified cells including cancer cells.

The term “companion diagnostic” used herein refers to a diagnostic test used as a companion to a therapeutic drug to determine its applicability to a specific person.

The term “antigen” used herein refers to a toxin or other foreign substance which induces an immune response in the body, especially the production of antibodies or a cellular response.

The term “Immunotherapy” used herein refers to a treatment to stimulate or restore the ability of the immune (defense) system to fight infection and disease.

The term “cannabinoid” used herein refers to any chemical in marijuana that causes drug-like effects all through the body, including the central nervous system and the immune system.

The term “CBD” used herein refers to a legal nonintoxicating cannabinoids found in cannabis and hemp.

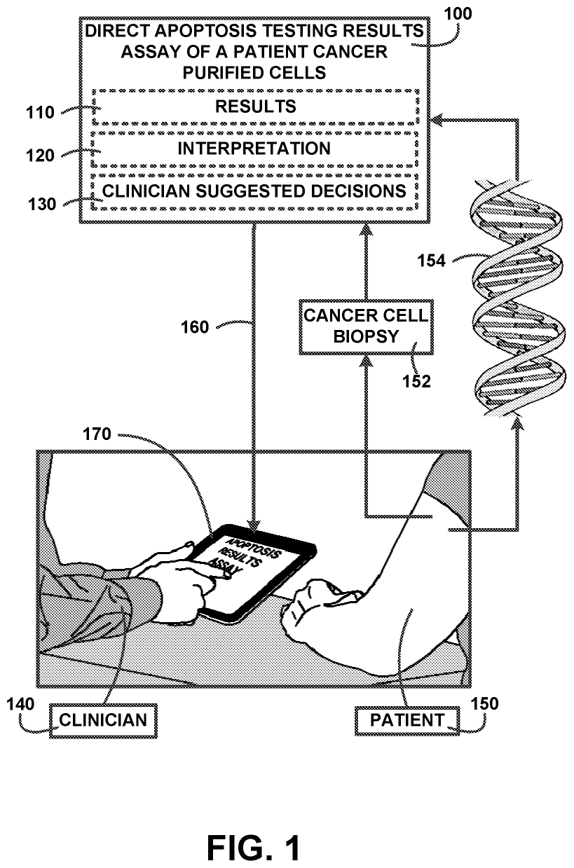

FIG. 1 shows for illustrative purposes only an example of an overview of a method and devices for direct apoptosis assay of purified cells of one embodiment. FIG. 1 shows a patient 150 providing a cancer cell biopsy 152 and DNA genomic testing 154. The method and devices for direct apoptosis assay of purified cells processes the cancer cell biopsy 152 and DNA genomic testing 154 provided by the patient 150. The cancer cell biopsy 152 tissues are processed in at least one cell purification procedure. The purified cells then are processed in a series of apoptosis next-generation sequence testing with selected drugs and combinations of drugs to determine which is the most effective in killing in this example the patient's cancer cells. Recommending part inhibitors as part of suggestions to doctors includes using the APOP assay with and/or without next generation sequencing, oral swabs and/or b mood in parallel to be able to assess where DNA mutations exists, for example in a tumor or also due to bloodline mutations.

The DNA genomic testing 154 is reviewed to identify genetic markers that show any variants in the genes that would affect the use of one or more drugs that could be used in a treatment regimen. Direct apoptosis testing results assay of a patient cancer purified cells 100 are correlated into results 110, interpretations 120 and clinician suggested decisions 130. The correlated apoptosis testing results assay including the results 110, interpretations 120 and clinician suggested decisions 130 are transmitted for example to a clinician digital tablet 160. The clinician digital tablet 160 displays the apoptosis testing results assay using an apoptosis application installed 170 on the clinician digital tablet 160. This allows the clinician 140 to review the results, interpretations and suggested decisions with the patient 150 for planning a treatment course of one embodiment.

### DETAILED DESCRIPTION

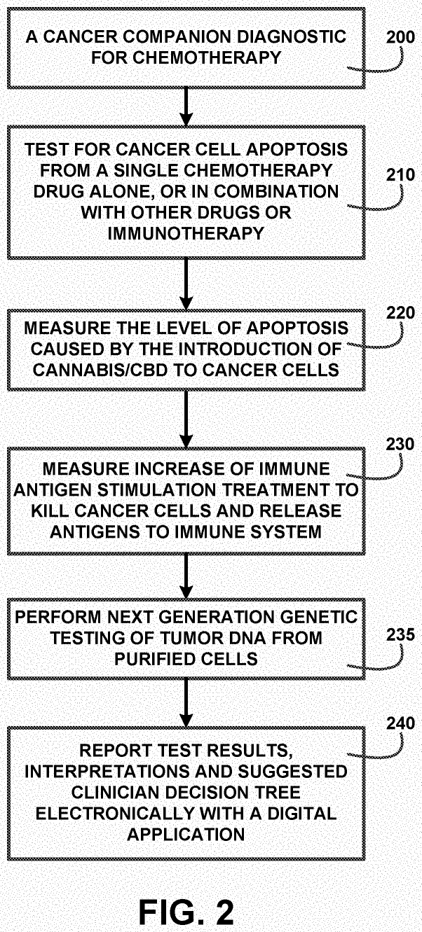

FIG. 2 shows a block diagram of an overview of a cancer companion diagnostic for chemotherapy of one embodiment. FIG. 2 shows a cancer companion diagnostic for chemotherapy 200 used to test for cancer cell apoptosis from a single chemotherapy drug alone, or in combination with other drugs or immunotherapy 210. The direct apoptosis testing results assay of a patient cancer purified cells 100 of FIG. 1 in one sequencing example of the results record a measure of the level of apoptosis caused by the introduction of cannabinoids/CBD to cancer cells 220. Also, measure increase of immune antigen stimulation treatment to kill cancer cells and release antigens to immune system 230. Perform next generation genetic testing of tumor DNA from purified cells 235. The direct apoptosis testing results assay of a patient cancer purified cells 100 of FIG. 1 is used to report test results, interpretations and suggested clinician decision tree electronically with a digital application 240 to make the data available to clinicians for reviewing with patients of one embodiment.

### Performing a Cancer Companion Diagnostic Direct Apoptosis Assay of Purified Cancer Cells:

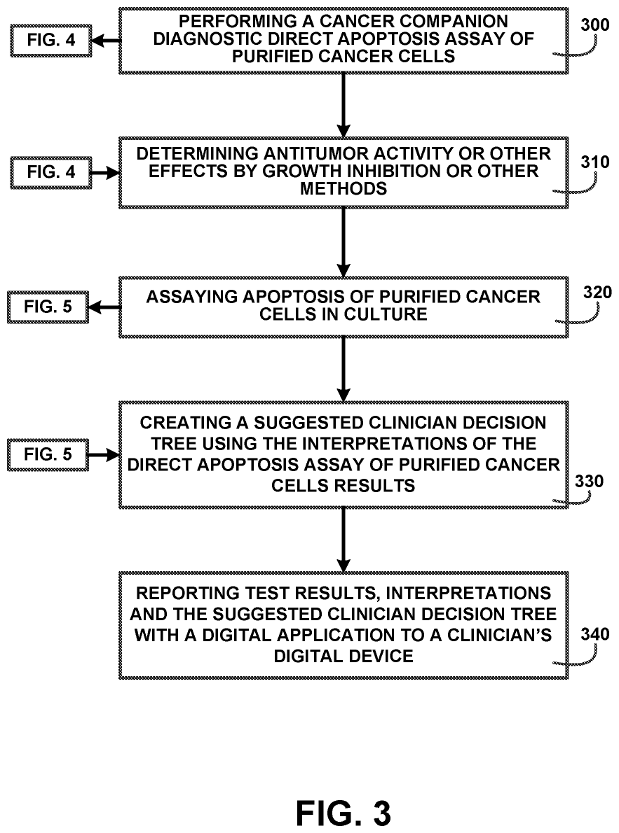

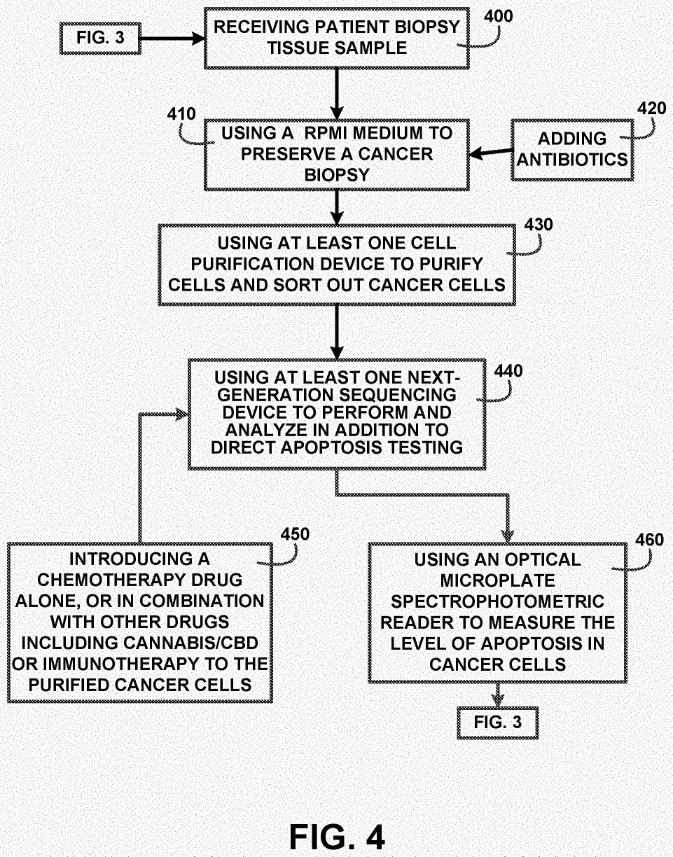

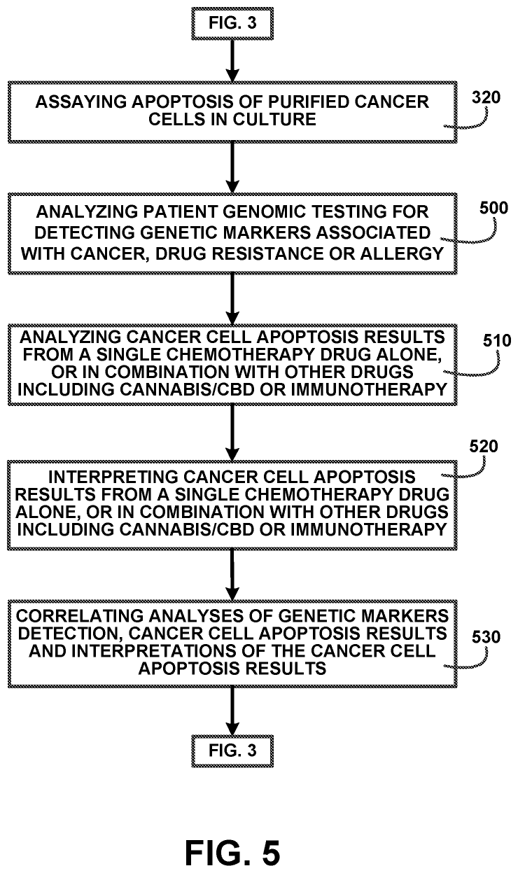

FIG. 3 shows a block diagram of an overview flow chart of performing a cancer companion diagnostic direct apoptosis assay of purified cancer cells of one embodiment. FIG. 3 shows performing a cancer companion diagnostic direct apoptosis assay of purified cancer cells 300 with descriptions of processes showing in FIG. 4. After performing a cancer companion diagnostic direct apoptosis assay of purified cancer cells 300 as shown in FIG. 4 are processes for determining antitumor activity or other effects by growth inhibition or other methods 310. Processing continues with assaying apoptosis of purified cancer cells in culture 320 with descriptions of processes showing in FIG. 5. After performing the processes for assaying apoptosis of purified cancer cells in culture 320 as shown in FIG. 5 the processing continues for creating a suggested clinician decision tree using the interpretations of the direct apoptosis assay of purified cancer cells results 330. The cancer companion diagnostic direct apoptosis assay of purified cancer cells includes reporting test results, interpretations and the suggested clinician decision tree with a digital application to a clinician's digital device 340 for allowing a clinician and patient to discuss a course of treatment based on the results of the testing for that specific patient of one embodiment.

### Receiving Patient Biopsy Tissue Sample:

FIG. 4 shows a block diagram of an overview flow chart of receiving patient biopsy tissue sample of one embodiment. FIG. 4 shows processes for the cancer companion diagnostic direct apoptosis assay of purified cancer cells 300 of FIG. 3 that include receiving patient biopsy tissue sample 400. The process includes using a RPMI medium or other medium with or without other additives to preserve a cancer biopsy 410. The process includes adding antibiotics 420 to a portion of the preserved cancel biopsy. Preparation of the cancer cells for testing includes using at least one cell purification device to purify cells and sort out cancer cells 430.

Individual tests on the cancer cells are performed using at least one next-generation sequencing device to perform can analyze in addition to direct apoptosis testing 440. The direct apoptosis testing includes introducing a chemotherapy drug alone, or in combination with other drugs including cannabinoids/CBD or immunotherapy to the purified cancer cells 450. The apoptosis effect of the chemotherapy drug alone, or in combination with other drugs including cannabinoids/CBD or immunotherapy on the purified cancer cells is determined using an optical microplate spectrophotometric reader to measure the level of apoptosis in cancer cells 460. After the determinations of the apoptosis affects the processing returns to FIG. 3 of one embodiment.

### Assaying Apoptosis of Purified Cancer Cells in Culture:

FIG. 5 shows a block diagram of an overview flow chart of assaying apoptosis of purified cancer cells in culture of one embodiment. FIG. 5 shows a continuation of processing from FIG. 3 that includes assaying apoptosis of purified cancer cells in culture 320. The process includes patient genomic testing using cells from the preserved cancer biopsy and may include analysis of patient blood sample. Effectiveness of various treatments may vary depending on the patient's genetic make-up. The assaying apoptosis processing may include analyzing patient genomic testing for detecting genetic markers associated with cancer, drug resistance or allergy 500 and in parallel to be able to assess where DNA mutations exists, for example in a tumor or also due to bloodline mutations.

Analyzing cancer cell apoptosis results from a single chemotherapy drug alone, or in combination with other drugs including cannabinoids/CBD or immunotherapy 510 identifies the potential success of a treatment for the single chemotherapy drug alone, or in combination with other drugs including cannabinoids/CBD or immunotherapy. Interpreting cancer cell apoptosis results from a single chemotherapy drug alone, or in combination with other drugs including cannabinoids/CBD or immunotherapy 520 assists a clinician in evaluating the testing results. Correlating analyses of genetic markers detection, cancer cell apoptosis results and interpretations of the cancer cell apoptosis results 530 is used in the processes following as described in FIG. 3 of one embodiment.

### A Method for Direct APOP Assay of Purified Cells:

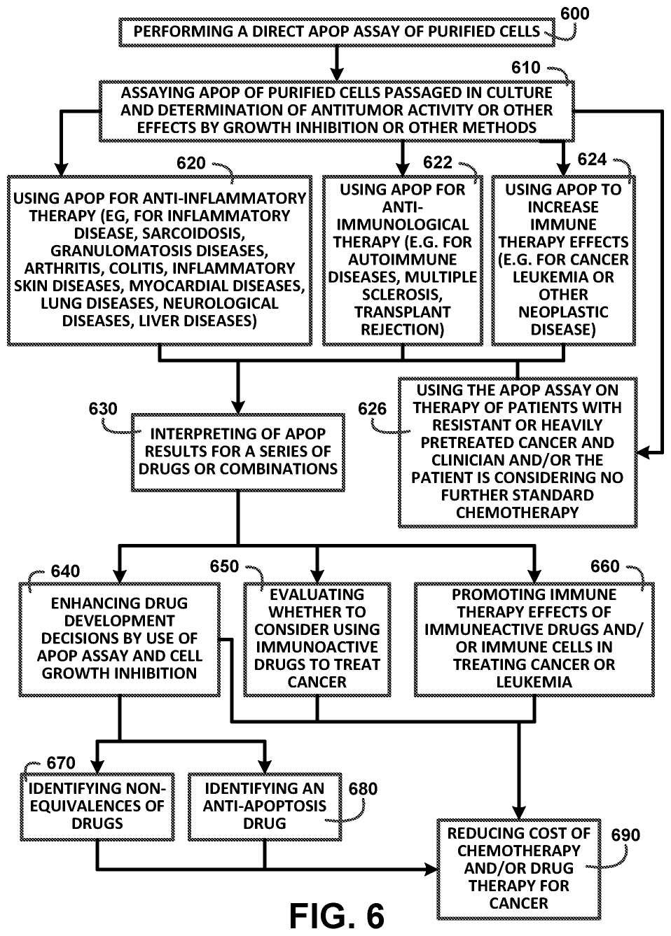

FIG. 6 shows a block diagram of an overview flow chart of a method for a direct APOP assay of purified cells of one embodiment. FIG. 6 shows a method for direct APOP assay of purified cells including performing a direct APOP assay of purified cells 600. The method for direct APOP assay of purified cells includes performing the assays on patient purified cells to assess the effectiveness of drug treatments specific to that patient's current condition including genetics and prior treatment affects. The performing a direct APOP assay of purified cells 600 includes assaying apoptosis of purified cells passaged in culture and determination of antitumor activity or other effects by growth inhibition or other methods 610. Using APOP for anti-inflammatory therapy (e.g., for inflammatory disease, sarcoidosis, granulomatosis diseases, arthritis, colitis, inflammatory skin diseases, myocardial diseases, lung diseases, neurological diseases, liver diseases) 620. Using APOP for anti-immunological therapy (e.g. for autoimmune diseases, multiple sclerosis, transplant rejection) 622. Using APOP to increase immune therapy effects (e.g. for cancer, leukemia or other neoplastic disease) 624. Using the APOP assay on therapy of patients with resistant or heavily pretreated cancer and clinician and/or the patient is considering no further standard chemotherapy 626.

The method for direct APOP assay of purified cells includes interpreting of APOP results for a series of drugs or combinations 630 for suggested clinician decisions in choosing potential treatments. The clinicians may receive the direct APOP assay and suggested clinician decisions using a direct APOP assay of purified cells application installed on a clinician's digital device including a smart phone, digital tablet and computer. The method for direct APOP assay of purified cells is used for enhancing drug development decisions by use of APOP assay and cell growth inhibition 640, identifying non-equivalences of drugs 670, identifying an anti-Apoptosis drug 680, evaluating whether to consider using immunoactive drugs to treat cancer 650, promoting immune therapy effects of immuno-active drugs and/or immune cells in treating cancer or leukemia 660 and reducing cost of chemotherapy and/or drug therapy for cancer 690 of one embodiment.

### 1.0 Direct APOP Assay of Purified Cells:

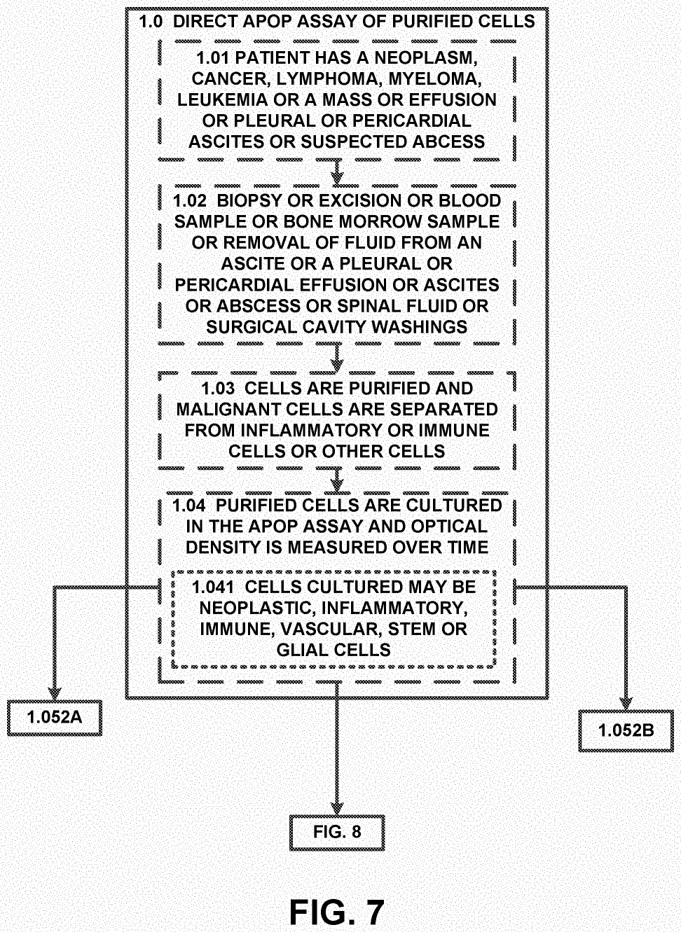

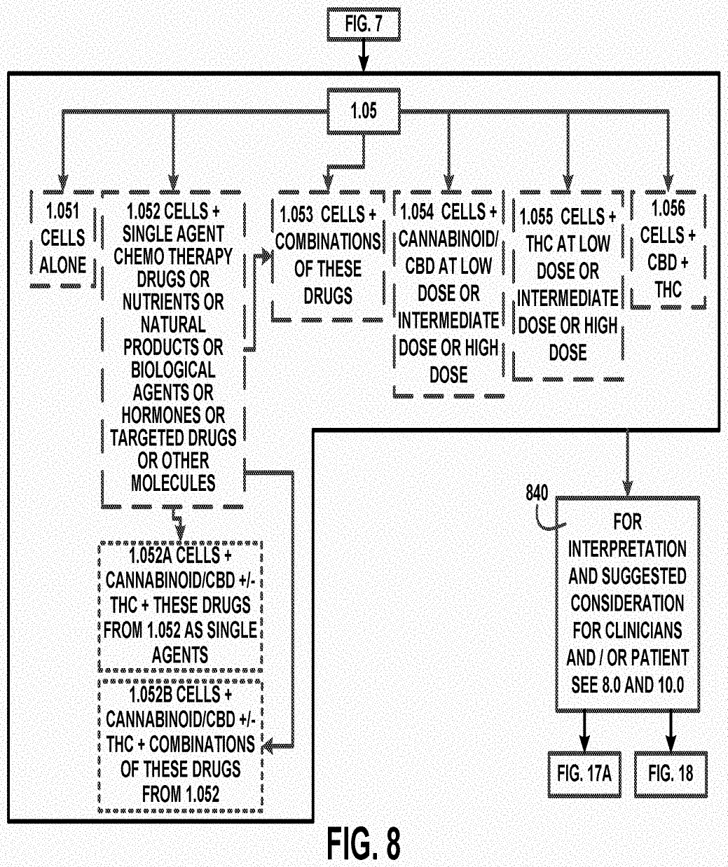

FIG. 7 shows a block diagram of an overview of 1.0 direct APOP assay of purified cells of one embodiment. FIG. 7 shows a method for direct APOP assay of purified cells step 1.0 direct APOP assay of purified cells 700. Step 1.0 direct APOP assay of purified cells 700 includes cells including 1.01 patient has a neoplasm, cancer, lymphoma, myeloma, leukemia or a mass or effusion or pleural or pericardial ascites or suspected abscess 710 and 1.02 biopsy or excision or blood sample or bone morrow sample or removal of fluid from an ascites or a pleural or pericardial effusion or ascites or abscess or spinal fluid or surgical cavity washings 720. 1.03 cells are purified and malignant cells are separated from inflammatory or immune cells or other cells 730. 1.04 purified cells are cultured in the APOP assay and optical density is measured over time 740. 1.041 cells cultured may be neoplastic, inflammatory, immune, vascular, stem or glial cells 750. The processes continue in 1.052a 760 and 1.052b 770 and are described in FIG. 8 of one embodiment.

### 1.05 Purified Cells+Drugs:

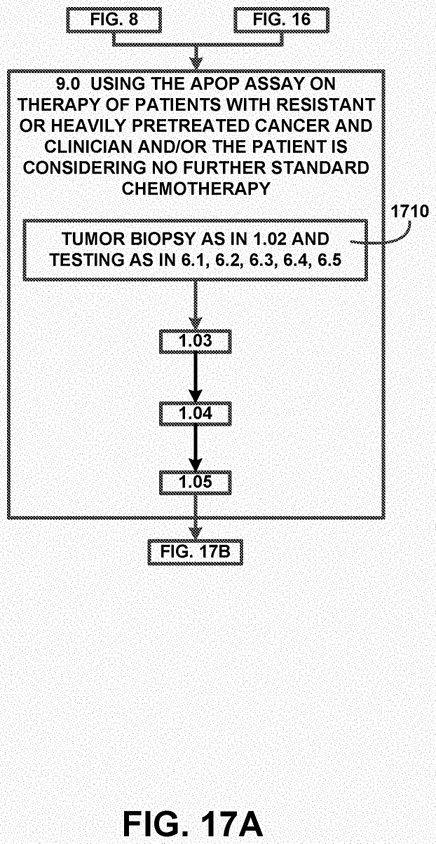

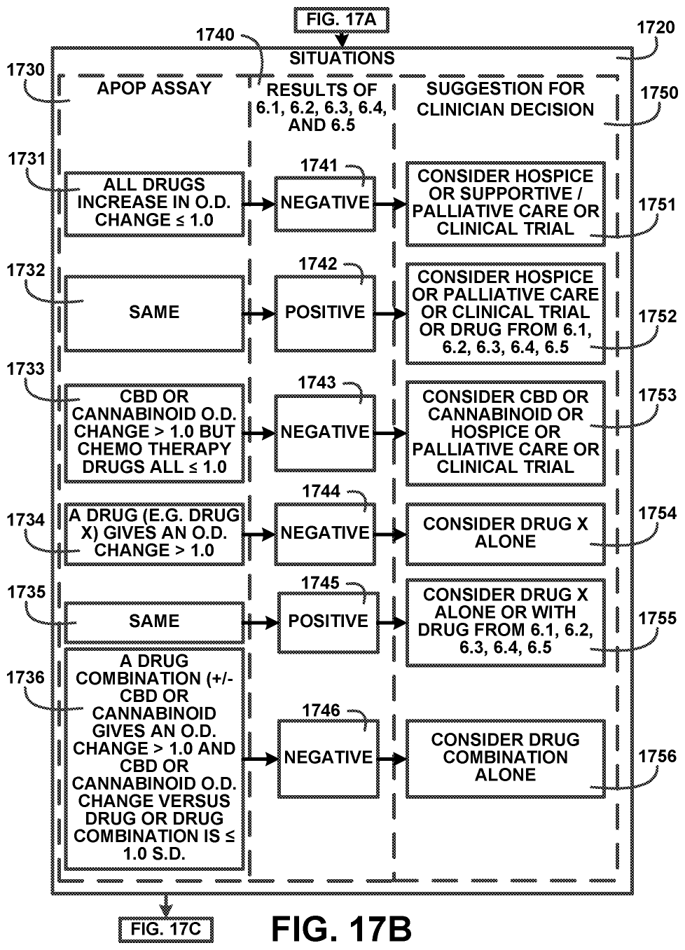

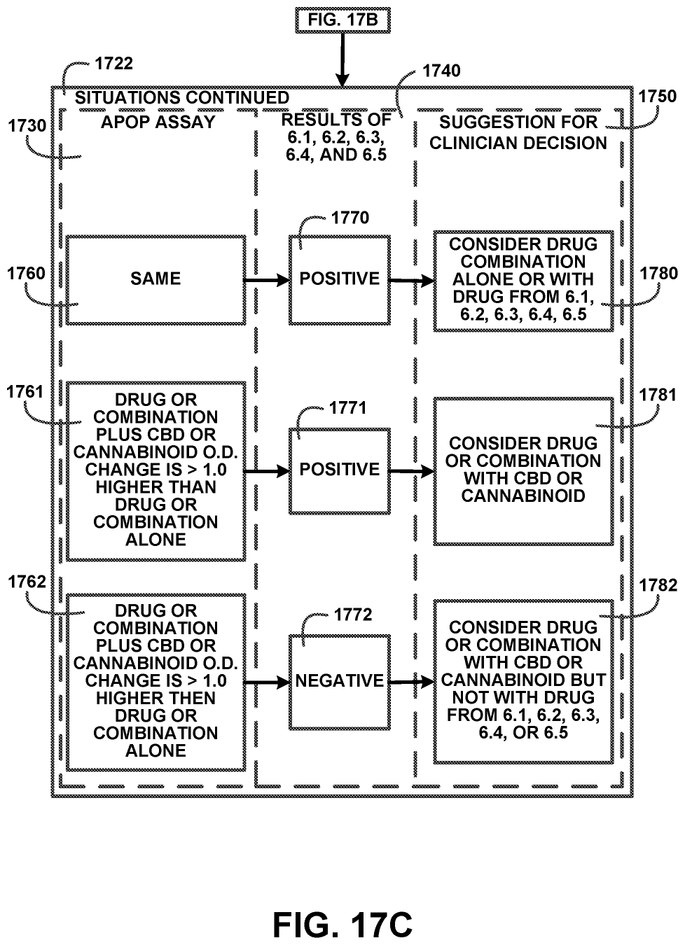

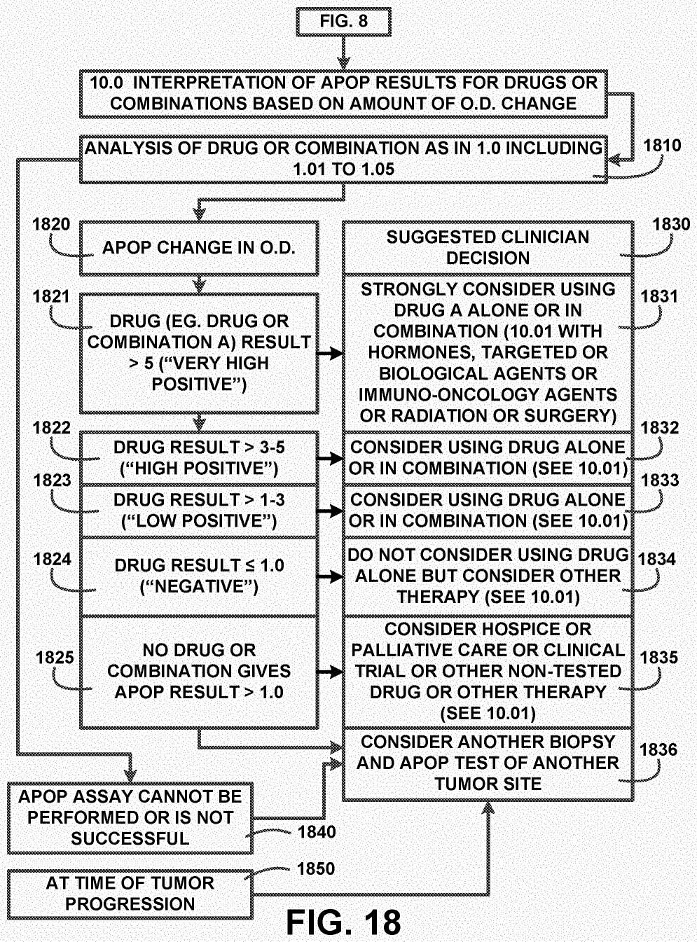

FIG. 8 shows a block diagram of an overview of 1.05 purified cells+drugs of one embodiment. FIG. 8 shows a continuation from FIG. 7 including step 1.05 800 using 1.051 cells alone 810 and 1.052 cells+single agent chemo therapy drugs or nutrients or natural products or biological agents or hormones or targeted drugs or other molecules 811. 1.052a cells+cannabinoid/CBD+/−THC+these drugs from 1.052 as single agents 820 and 1.052b cells+cannabinoid/CBD+/−THC+combinations of these drugs from 1.052 830. The processes continue with 1.053 cells+combinations of these drugs 812, 1.054 cells+cannabinoid/CBD at low dose or intermediate dose or high dose 813, 1.055 cells+THC at low dose or intermediate dose or high dose 814 and 1.056 cells+CBD+THC 815. The results are assessed for interpretation and suggested consideration for clinicians and/or patient see 8.0 and 10.0 840. Additional descriptions are shown in FIG. 17A and FIG. 18 of one embodiment.

### 1.1 Culture and Determination of Antitumor Activity:

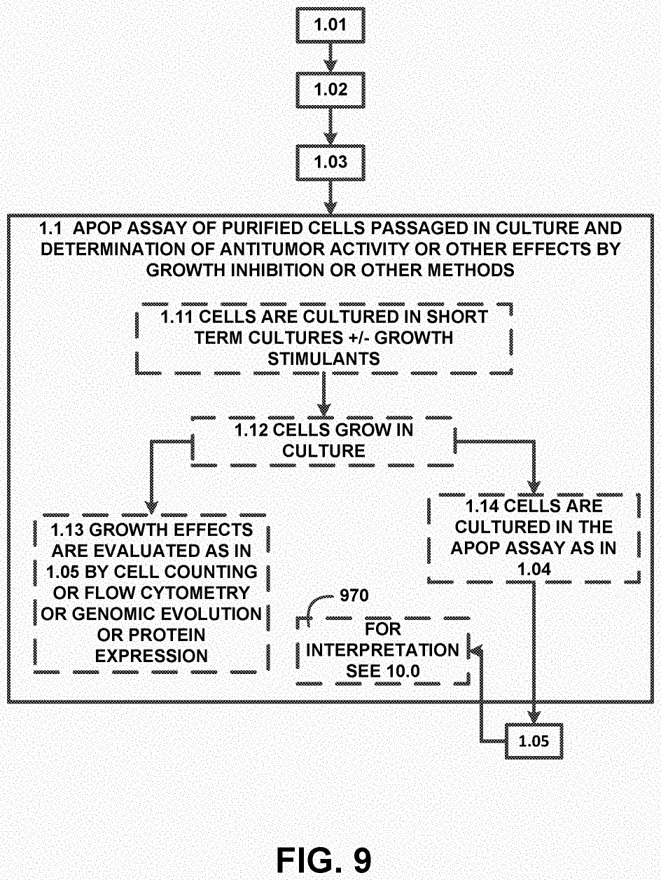

FIG. 9 shows a block diagram of an overview of 1.1 culture and determination of antitumor activity of one embodiment. FIG. 9 shows a continuation of steps 1.01 900, 1.02 902 and 1.03 904 with 1.1 APOP assay of purified cells passaged in culture and determination of antitumor activity or other effects by growth inhibition or other methods 910. 1.11 cells are cultured in short term cultures+/−growth stimulants 920 and 1.12 cells grow in culture 930. 1.13 growth effects are evaluated as in 1.05 by cell counting or flow cytometry or genomic evolution or protein expression 940. 1.14 cells are cultured in the APOP assay as in 1.04 950 and processed in 1.05 300 and for interpretation see 10.0 970 of one embodiment.

### 2.0 Using APOP for Anti-Inflammatory Therapy:

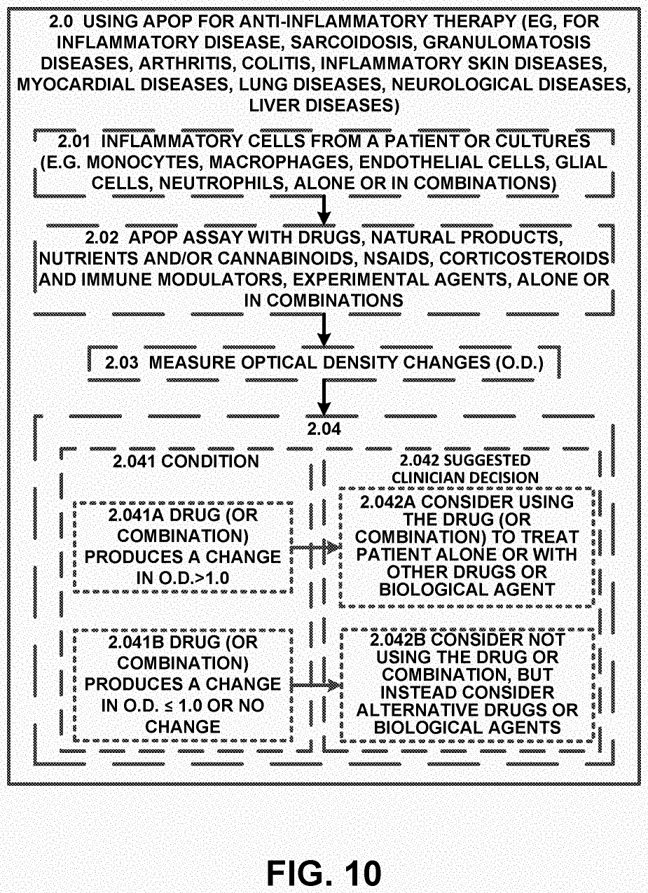

FIG. 10 shows a block diagram of an overview of 2.0 using APOP for anti-inflammatory therapy of one embodiment. FIG. 10 shows step 2.0 using APOP for anti-inflammatory therapy (e.g., for inflammatory disease, sarcoidosis, granulomatosis diseases, arthritis, colitis, inflammatory skin diseases, myocardial diseases, lung diseases, neurological diseases, liver diseases) 1000. 2.01 inflammatory cells from a patient or cultures (e.g. monocytes, macrophages, endothelial cells, glial cells, neutrophils, alone or in combinations) 1010. Processing includes 2.02 APOP assay with drugs, natural products, nutrients and/or cannabinoids, NSAIDs, corticosteroids and immune modulators, experimental agents, alone or in combinations 1020. Testing evaluation include step 2.03 measure optical density changes (O.D.) 1030. The step 2.03 measure optical density changes (O.D.) 1030 evaluations include 2.04 1040 which are correlated using a 2.041 condition 1050, 2.041a drug (or combination) produces a change in O.D.>1.0 1052 and 2.042 suggested clinician decision 1060. For example 2.042a consider using the drug (or combination) to treat patient alone or with other drugs or biological agent 1062. 2.041b drug (or combination) produces a change in O.D.≤1.0 or no change 1054 and 2.042b consider not using the drug or combination, but instead consider alternative drugs or biological agents 1064 of one embodiment.

### 3.0 Using APOP for Anti-Immunological Therapy:

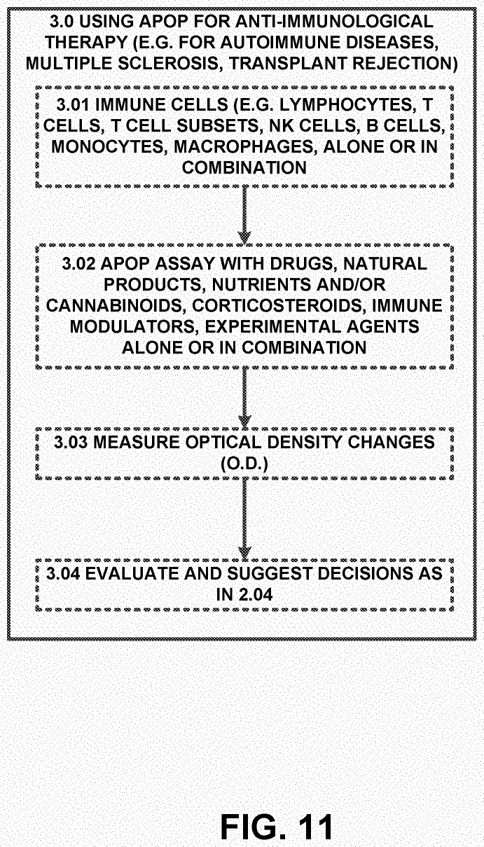

FIG. 11 shows a block diagram of an overview of 3.0 using APOP for anti-immunological therapy of one embodiment. FIG. 11 shows step 3.0 using APOP for anti-immunological therapy (e.g. for autoimmune diseases, multiple sclerosis, transplant rejection) 1100 using 3.01 immune cells (e.g. lymphocytes, T cells, T cell subsets, NK cells, B cells, monocytes, macrophages, alone or in combination 1110. The process includes 3.02 APOP assay with drugs, natural products, nutrients and/or cannabinoids, corticosteroids, immune modulators, experimental agents alone or in combination 1120. This process include 3.03 measure optical density changes (O.D.) 1130 and 3.04 evaluate and suggest decisions as in 2.04 1140 of one embodiment.

### 4.0 Using APOP to Increase Immune Therapy Effects:

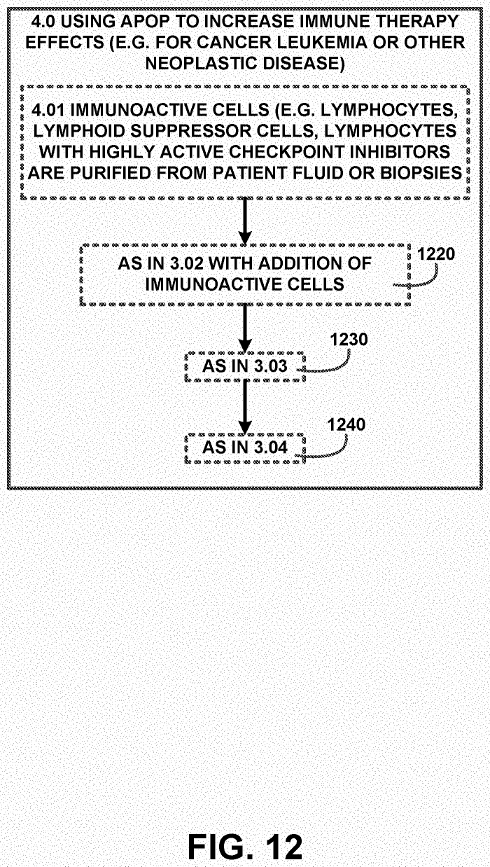

FIG. 12 shows a block diagram of an overview of 4.0 using APOP to increase immune therapy effects of one embodiment. FIG. 12 shows step 4.0 using APOP to increase immune therapy effects (e.g. for cancer leukemia or other neoplastic disease) 1200. 4.01 immunoactive cells (e.g. lymphocytes, lymphoid suppressor cells, lymphocytes with highly active checkpoint inhibitors are purified from patient fluid or biopsies 1210 and as in 3.02 with addition of immunoactive cells 1220, as in 3.03 1230 and as in 3.04 1240 of one embodiment.

### 5.0 Extended APOP Assay Decision Tree:

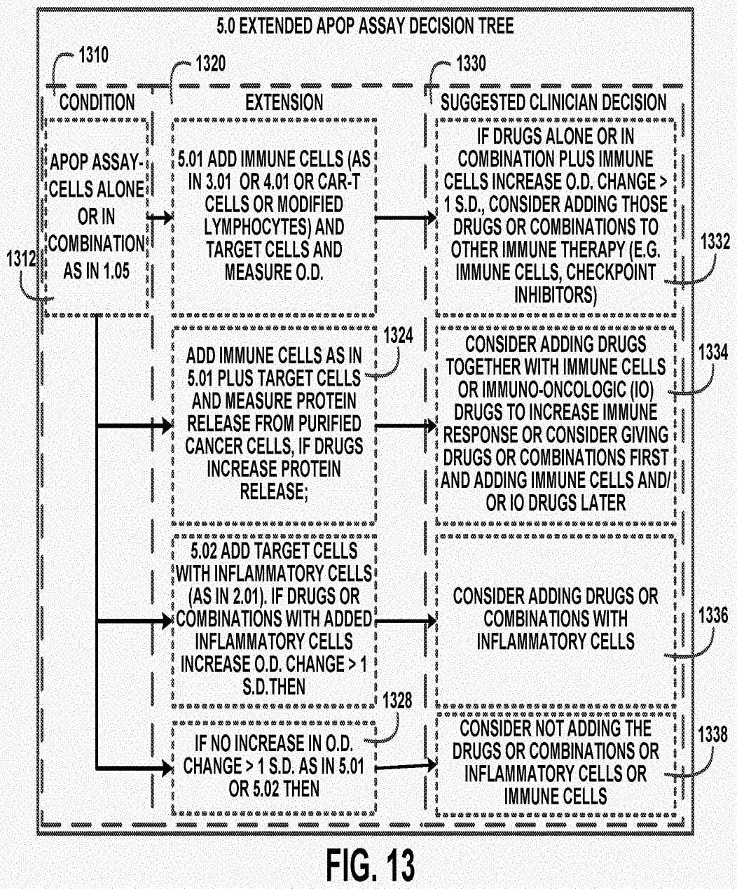

FIG. 13 shows a block diagram of an overview of 5.0 extended APOP assay decision tree of one embodiment. FIG. 13 shows 5.0 extended APOP assay decision tree 1300. The 5.0 extended APOP assay decision tree 1300 includes a correlation of condition 1310, extension 1320 and suggested clinician decision 1330. A condition 1310 includes for example APOP assay-cells alone or in combination as in 1.05 1312, an extension 1320 for example 5.01 add immune cells (as in 3.01 or 4.01 or car-t cells or modified lymphocytes) and target cells and measure O.D. 1322 and suggested clinician decision 1330 for example if drugs alone or in combination plus immune cells increase O.D. change >1 S.D., consider adding those drugs or combinations to other immune therapy (e.g. immune cells, checkpoint inhibitors) 1332. The 5.0 extended APOP assay decision tree 1300 continues with same 1314, add immune cells as in 5.01 plus target cells and measure protein release from purified cancer cells, if drugs increase protein release; 1324 and consider adding drugs together with immune cells or immuno-oncologic (IO) drugs to increase immune response or consider giving drugs or combinations first and adding immune cells and/or IO drugs later 1334. Same 1316 condition 1310, 5.02 add target cells with inflammatory cells (as in 2.01). If drugs or combinations with added inflammatory cells increase O.D. change >1 S.D. then 1326, and consider adding drugs or combinations with inflammatory cells 1336. Same 1318 condition 1310, if no increase in O.D. change >1 S.D. as in 5.01 or 5.02 then 1328, consider not adding the drugs or combinations or inflammatory cells or immune cells 1338 of one embodiment.

### 6.0 Pre-APOP Assay Decision Tree:

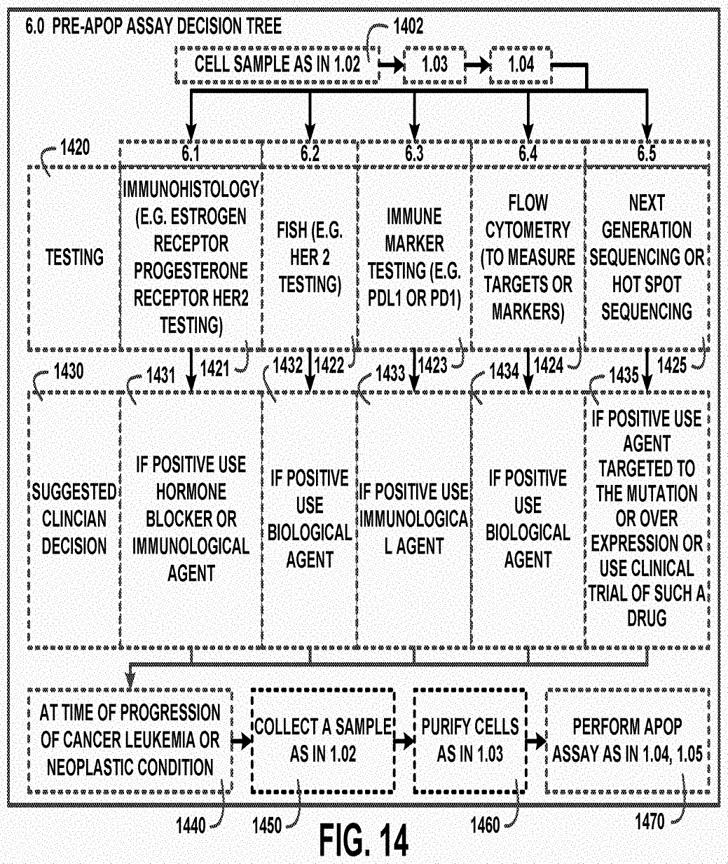

FIG. 14 shows a block diagram of an overview of 6.0 pre-APOP assay decision tree of one embodiment. FIG. 14 shows step 6.0 pre-APOP assay decision tree 1400 using a cell sample as in 1.02 1402 followed by 1.03 404 and 1.04 1406. The 6.0 pre-APOP assay decision tree 1400 includes testing 1420 and suggested clinician decision 1430 for the series of testing conditions for example 6.1 1410, immunohistology (e.g. estrogen receptor progesterone receptor her2 testing) 1421 and if positive use hormone blocker or immunological agent 1431.

Additional testing conditions include 6.2 1412, fish (e.g. her 2 testing) 1422, if positive use biological agent 1432; 6.3 1414, immune marker testing (e.g. pdl1 or pd1) 1423, and if positive use immunological agent 1433; 6.4 1416, flow cytometry (to measure targets or markers) 1424, and if positive use biological agent 1420; 6.5 1418, next generation sequencing or hot spot sequencing 825, and if positive use agent targeted to the mutation or over expression or use clinical trial of such a drug 1435. Additional suggested clinician decision 1430 include at time of progression of cancer leukemia or neoplastic condition 1440, collect a sample as in 1.02 1450, purify cells as in 1.03 1460, and perform APOP assay as in 1.04, 1.05 1470 of one embodiment.

### 7.0 Parallel APOP Assay Decision Tree:

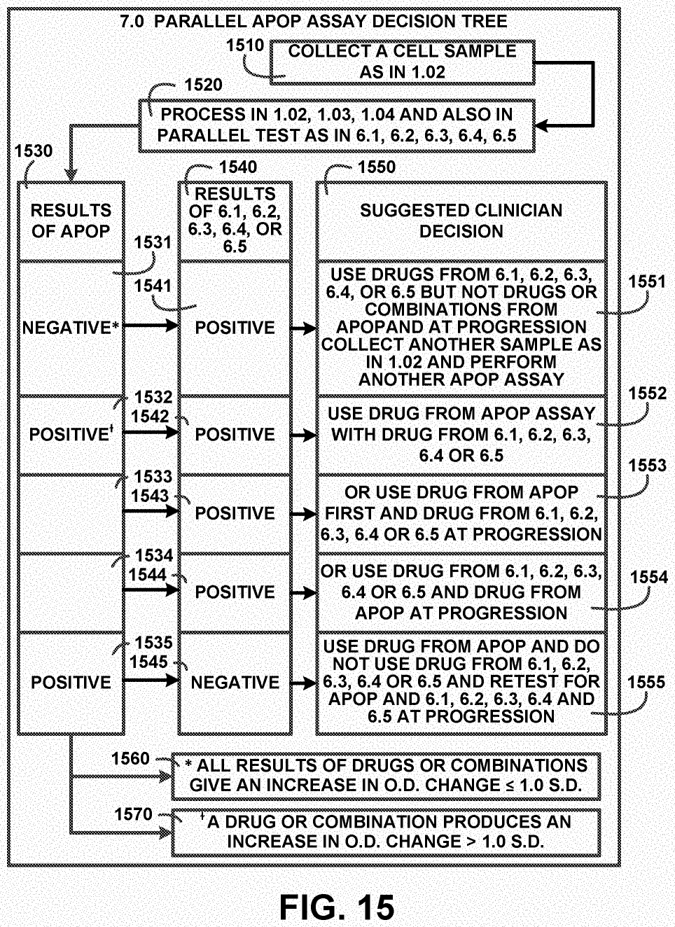

FIG. 15 shows a block diagram of an overview of 7.0 parallel APOP assay decision tree of one embodiment. FIG. 15 shows 7.0 parallel APOP assay decision tree 1500 with steps that include collect a cell sample as in 1.02 1510, process in 1.02, 1.03, 1.04 and also in parallel test as in 6.1, 6.2, 6.3, 6.4, 6.5 1520. The 7.0 parallel APOP assay decision tree 1500 correlates the results of APOP 1530, results of 6.1, 6.2, 6.3, 6.4, or 6.5 1540 and suggested clinician decision 1550 for example negative* 1531 wherein * all results of drugs or combinations give an increase in O.D. change ≤1.0 S.D. 1560, positive 1541, and use drugs from 6.1, 6.2, 6.3, 6.4, or 6.5 but not drugs or combinations from APOP and at progression collect another sample as in 1.02 and perform another APOP assay 1551. Another example for positive⁺1532 wherein ⁺ a drug or combination produces an increase in O.D. change >1.0 S.D. 1570, positive 1542, and use drug from APOP assay with drug from 6.1, 6.2, 6.3, 6.4 or 6.5 1552.

Block 1533 and 1534 are empty and reflect the same results of APOP 1530 shown in positive+ 1532, with positive 1543 and or use drug from APOP first and drug from 6.1, 6.2, 6.3, 6.4 or 6.5 at progression 1553. Continuing with positive 1544, and or use drug from 6.1, 6.2, 6.3, 6.4 or 6.5 and drug from APOP at progression 1554. Results of APOP 1530 show positive 1535, negative 1545 and use drug from APOP and do not use drug from 6.1, 6.2, 6.3, 6.4 or 6.5 and retest for APOP and 6.1, 6.2, 6.3, 6.4 and 6.5 at progression 1555 of one embodiment.

### 8.0 Interpretation of APOP Results for a Series of Drugs or Combinations:

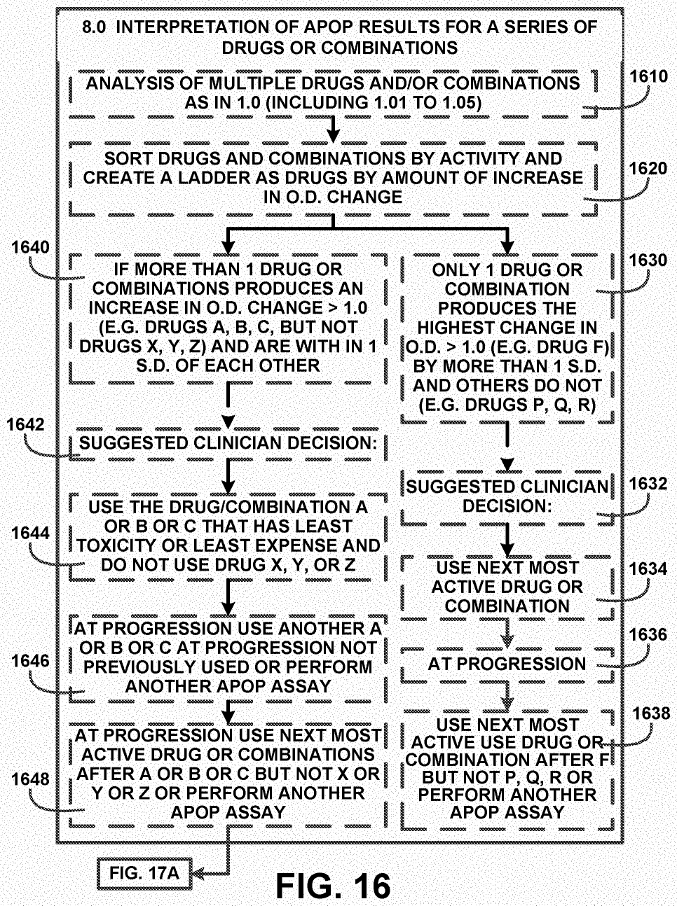

FIG. 16 shows a block diagram of an overview of 8.0 interpretation of APOP results for a series of drugs or combinations of one embodiment. FIG. 16 shows step 8.0 interpretation of APOP results for a series of drugs or combinations 1600. Step 8.0 interpretation of APOP results for a series of drugs or combinations 1600 includes an analysis of multiple drugs and/or combinations as in 1.0 (including 1.01 to 1.05) 1610 and steps to sort drugs and combinations by activity and create a ladder as drugs by amount of increase in O.D. change 1620. For example if more than 1 drug or combinations produces an increase in O.D. change >1.0 (e.g. drugs A, B, C, but not drugs X, Y, Z) and are within 1 S.D. of each other 1640 a suggested clinician decision: 1642 includes use the drug/combination A or B or C that has least toxicity or least expense and do not use drug X, Y, or Z 1644, at progression use another A or B or C at progression not previously used or perform another APOP assay 1646 and at progression use next most active drug or combinations after A or B or C but not X or Y or Z or perform another APOP assay 1648 and continue to FIG. 17A of one embodiment.

Another example includes only 1 drug or combination produces the highest change in O.D.>1.0 (e.g. drug F) and by more than 1 S.D. and others do not (e.g. drugs P, Q, R) 1630. A suggested clinician decision: 1632 includes use next most active drug or combination 1634, at progression 1636 and use next most active use drug or combination after F but not P, Q, R or perform another APOP assay 1638 of one embodiment.

9.0 Using the APOP Assay on Therapy of Patients with Resistant or Heavily Pretreated Cancer:

FIG. 17A shows a block diagram of an overview of 9.0 using the APOP assay on therapy of patients with resistant or heavily pretreated cancer of one embodiment. FIG. 17A shows a continuation from FIG. 8 and FIG. 16 including step 9.0 using the APOP assay on therapy of patients with resistant or heavily pretreated cancer and clinician and/or the patient is considering no further standard chemotherapy 1700. Step 9.0 using the APOP assay on therapy of patients with resistant or heavily pretreated cancer and clinician and/or the patient is considering no further standard chemotherapy 1700 includes processing with a tumor biopsy as in 1.02 and testing as in 6.1, 6.2, 6.3, 6.4, 6.5 1710 followed by 1.03 404, 1.04 906, and 1.05 300. The steps are further described in FIG. 17B of one embodiment.

### Situations:

FIG. 17B shows a block diagram of an overview of situations of one embodiment. FIG. 17B shows a continuation from FIG. 17A with situations 1720. Situations 1720 include APOP assay 1730, results of 6.1, 6.2, 6.3, 6.4, and 6.5 1740, and suggestion for clinician decision 1750. For example APOP assay 1730 includes all drugs increase in O.D. change 1.0 1731, negative 1741 and consider hospice or supportive/palliative care or clinical trial 1751. APOP assay 1730 is same 1732 with a positive 1742 results of 6.1, 6.2, 6.3, 6.4, and 6.5 1740 and consider hospice or palliative care or clinical trial or drug from 6.1, 6.2, 6.3, 6.4, 6.5 1752. The situations 1720 continue with CBD or cannabinoid O.D. change >1.0 but chemo therapy drugs all 1.0 1733, negative 1743, and consider CBD or cannabinoid or hospice or palliative care or clinical trial 1753. A drug (e.g. drug X) gives an O.D. change >1.0 1734, negative 1744, consider drug x alone 1754. Same 1735 APOP assay 1730, positive 1745, and consider drug X alone or with drug from 6.1, 6.2, 6.3, 6.4, 6.5 1755. A drug combination (+/−CBD or cannabinoid gives an O.D. change >1.0 and CBD or cannabinoid O.D. change versus drug or drug combination is <=1.0 S.D. 1736, negative 1746, and consider drug combination alone 1756 of one embodiment. The process continues in FIG. 17C.

### Situations Continued:

FIG. 17C shows a block diagram of an overview of situations continued of one embodiment. FIG. 17C shows a continuation from FIG. 17B with situations continued 1722 that include the APOP assay 1730, the results of 6.1, 6.2, 6.3, 6.4, and 6.5 1740 and the suggestion for clinician decision 1750. Examples include same 1760, positive 1770, and consider drug combination alone or with drug from 6.1, 6.2, 6.3, 6.4, 6.5 1780. An APOP assay 1730 with drug or combination plus CBD or cannabinoid O.D. change is >1.0 S.D. higher than drug or combination alone 1761, positive 1771, and consider drug or combination with CBD or cannabinoid 1781. Drug or combination plus CBD or cannabinoid O.D. change is >1.0 S.D. higher than drug or combination alone 1762, negative 1772, and consider drug or combination with CBD or cannabinoid but not with drug from 6.1, 6.2, 6.3, 6.4, or 6.5 1782 of one embodiment.

### 10.0 Interpretation of APOP Results for Drugs or Combinations Based on Amount of O.D. Change:

FIG. 18 shows a block diagram of an overview of 10.0 interpretation of APOP results for drugs or combinations based on amount of O.D. change of one embodiment. FIG. 18 shows a continuation from FIG. 8 with step 10.0 interpretation of APOP results for drugs or combinations based on amount of O.D. change 1800 with an analysis of drug or combination as in 1.0 including 1.01 to 1.05 1810. The analysis of drug or combination as in 1.0 including 1.01 to 1.05 1810 includes APOP change in O.D. 1820 and suggested clinician decision 1830. For example drug (e.g. drug or combination A) result >5 (“very high positive”) 1821 and strongly consider using drug A alone or in combination (10.01 with hormones, targeted or biological agents or immuneoncology agents or radiation or surgery) 1831.

Drug result >3-5 (“high positive”) 1822 with suggested clinician decision 1830 consider using drug alone or in combination (see 10.01) 1832. Drug result >1-3 (“low positive”) 1823 and consider using drug alone or in combination (see 10.01) 1833. Drug result 1.0 (“negative”) 1824 and do not consider using drug alone but consider other therapy (see 10.01) 1834. No drug or combination gives APOP result >1.0 1825 and consider hospice or palliative care or clinical trial or other non-tested drug or other therapy (see 10.01) 1835 and consider another biopsy and APOP test of another tumor site 1836. The analysis of drug or combination as in 1.0 including 1.01 to 1.05 1810 includes APOP assay cannot be performed or is not successful 1840 and consider another biopsy and APOP test of another tumor site 1836. Another situation includes at time of tumor progression 1850 consider another biopsy and APOP test of another tumor site 1836 of one embodiment.

11.0 Interpretation of APOP Results for Drugs with Similar Mechanisms of Action:

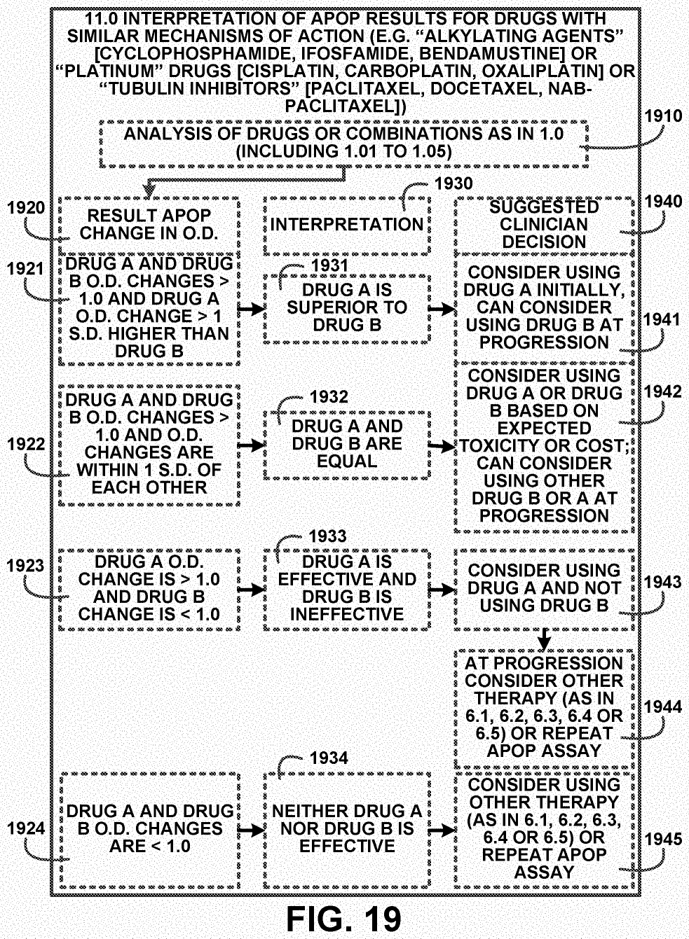

FIG. 19 shows a block diagram of an overview of 11.0 interpretation of APOP results for drugs with similar mechanisms of action of one embodiment. FIG. 19 shows 11.0 interpretation of APOP results for drugs with similar mechanisms of action (e.g. “alkylating agents” [cyclophosphamide, ifosfamide, bendamustine] or “platinum” drugs [cisplatin, carboplatin, oxaliplatin] or “tubulin inhibitors” [paclitaxel, docetaxel, nab-paclitaxel]) 1900 and includes an analysis of drugs or combinations as in 1.0 (including 1.01 to 1.05) 1910. The analysis of drugs or combinations as in 1.0 (including 1.01 to 1.05) 1910 is correlated in result APOP change in O.D. 1920, interpretation 1930, and suggested clinician decision 1940 categories. For example drug A and drug B O.D. changes >1.0 and drug A O.D. change >1 S.D. higher than drug B 1921 with the interpretation 1930 drug A is superior to drug B 1931, and consider using drug A initially, can consider using drug B at progression 1941.

Drug A and drug B O.D. changes >1.0 and O.D. changes are within 1 S.D. of each other 1922, drug A and drug B are equal 1932, and consider using drug A or drug B based on expected toxicity or cost; can consider using other drug B or A at progression 1942. Drug A O.D. change is >1.0 and drug B change is <1.0 1923, drug A is effective and drug B is ineffective 1933, consider using drug A and not using drug B 1943, and at progression consider other therapy (as in 6.1, 6.2, 6.3, 6.4 or 6.5) or repeat APOP assay 1944. Drug A and drug B O.D. changes are <1.0 1924, neither drug A nor drug B is effective 1934, and consider using other therapy (as in 6.1, 6.2, 6.3, 6.4 or 6.5) or repeat APOP assay 1945 of one embodiment.

12.0 Advanced Interpretation of APOP Results Using O.D. Change and Maximum O.D. Increase from a Single Drug or Combination:

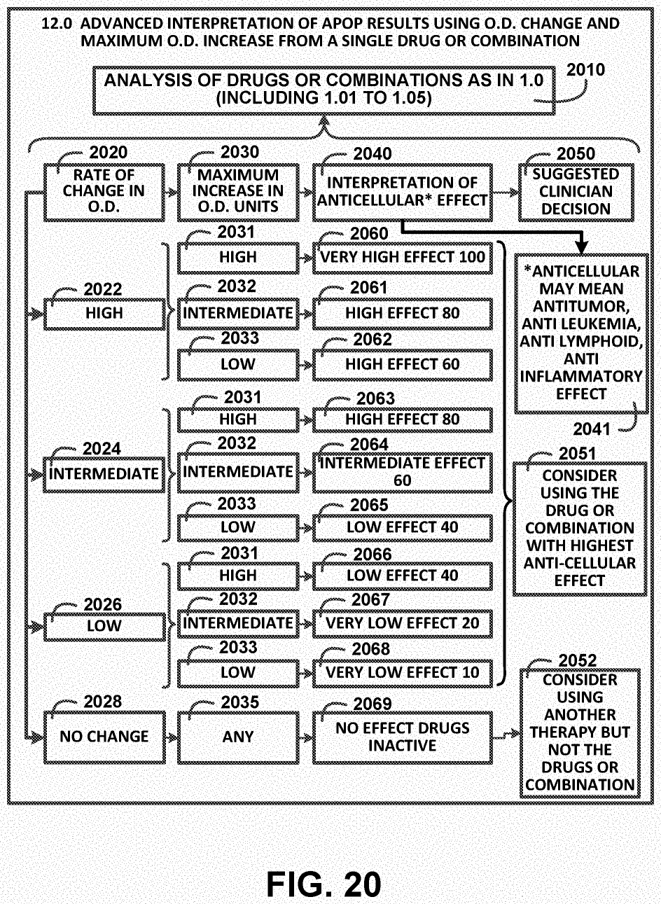

FIG. 20 shows a block diagram of an overview of 12.0 advanced interpretation of APOP results using O.D. change and maximum O.D. increase from a single drug or combination of one embodiment. FIG. 20 shows step 12.0 advanced interpretation of APOP results using O.D. change and maximum O.D. increase from a single drug or combination 2000 including an analysis of drugs or combinations as in 1.0 (including 1.01 to 1.05) 2010. The analysis of drugs or combinations as in 1.0 (including 1.01 to 1.05) 2010 a rate of change in O.D. 2020, maximum increase in O.D. units 2030, interpretation of anticellular* effect 2040 wherein *anticellular may mean antitumor, anti-leukemia, anti-lymphoid, anti-inflammatory effect 2041, and suggested clinician decision 2050 of one embodiment.

The rate of change in O.D. 2020 includes for example at least four rates of change in O.D. ratings including a high 2022, intermediate 2024, low 2026 and no change 2028. The high 2022, intermediate 2024, low 2026 rates each include a subset of rates for high, intermediate, and low. For example rate of change in O.D. 2020 high 2022, high 2031, high effect 80 2060; intermediate 2032, high effect 80 2061, and low 2033, high effect 60 2062 with suggested clinician decision 2050 consider using the drug or combination with highest anti-cellular effect 2051 of one embodiment.

Rate of change in O.D. 2020 intermediate 2024, high 2031, high effect 80 2063; intermediate 2032, intermediate effect 60 2064; low 2033, low effect 40 2065 and consider using the drug or combination with highest anti-cellular effect 2051 of one embodiment.

Rate of change in O.D. 2020 low 2026, high 2031, low effect 40 2066; intermediate 2032, very low effect 20 2067; low 2033, very low effect 10 2068 and consider using the drug or combination with highest anti-cellular effect 2051 of one embodiment.

Rate of change in O.D. 2020 no change 2028, any 2035, no effect drugs inactive 2069 and consider using another therapy but not the drugs or combination 2052 of one embodiment.

### 13.0 Enhancing Drug Development Decisions by Use of APOP Assay and Cell Growth Inhibition:

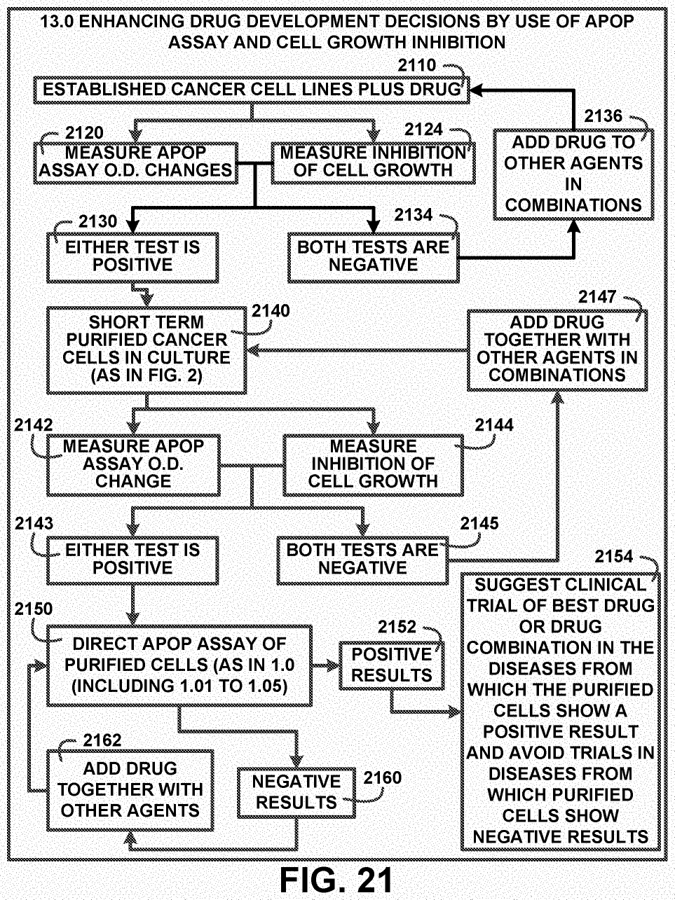

FIG. 21 shows a block diagram of an overview of 13.0 enhancing drug development decisions by use of APOP assay and cell growth inhibition of one embodiment. FIG. 21 shows 13.0 enhancing drug development decisions by use of APOP assay and cell growth inhibition 2100 with established cancer cell lines plus drug 2110. The 13.0 enhancing drug development decisions by use of APOP assay and cell growth inhibition 2100 combines processes to measure APOP assay O.D. changes 2120 and measure inhibition of cell growth 2124. Should the measurements show both tests are negative 2134 then add drug to other agents in combinations 2136 of one embodiment.

When either test is positive 2130 proceed with short term purified cancer cells in culture (as in FIG. 2) 2140. Measure APOP assay O.D. change 2142 and measure inhibition of cell growth 2144 and if both tests are negative 2145 add drug together with other agents in combinations 2147. If either test is positive 2143 direct APOP assay of purified cells (as in 1.0 (including 1.01 to 1.05) 2150. If positive results 2152 then suggest clinical trial of best drug or drug combination in the diseases from which the purified cells show a positive result and avoid trials in diseases from which purified cells show negative results 2154. If negative results 2160 then add drug together with other agents 2162 of one embodiment.

14.0 a Method to Reduce Cost of Chemotherapy and/or Drug Therapy for Cancer:

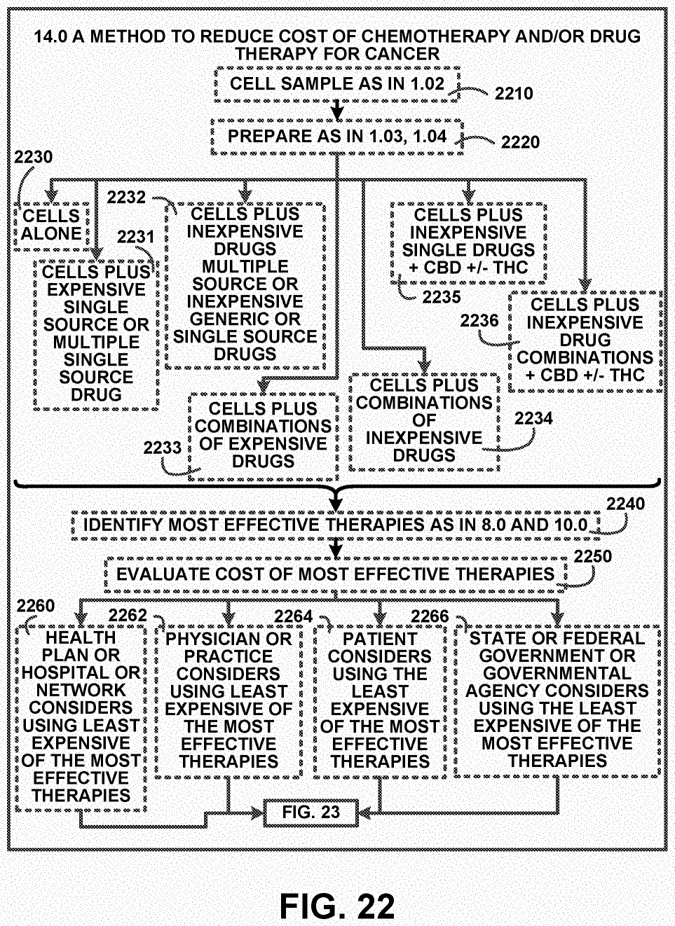

FIG. 22 shows a block diagram of an overview of 14.0 a method to reduce cost of chemotherapy and/or drug therapy for cancer of one embodiment. FIG. 22 shows step 14.0 a method to reduce cost of chemotherapy and/or drug therapy for cancer 2200. The 14.0 method to reduce cost of chemotherapy and/or drug therapy for cancer 2200 includes a cell sample as in 1.02 2210 and processing to prepare as in 1.03, 1.04 2220. The processing to prepare as in 1.03, 1.04 2220 includes cells alone 2230, cells plus expensive single source or multiple single source drug 2231, cells plus inexpensive drugs multiple source or inexpensive generic or single source drugs 2232, cells plus combinations of expensive drugs 2233, cells plus combinations of inexpensive drugs 2234, cells plus inexpensive single drugs+CBD+/−THC 2235, and cells plus inexpensive drug combinations+CBD+/−THC 2236.

The 14.0 method to reduce cost of chemotherapy and/or drug therapy for cancer 2200 includes a process to identify most effective therapies as in 8.0 and 10.0 2240 and a process to evaluate cost of most effective therapies 2250.

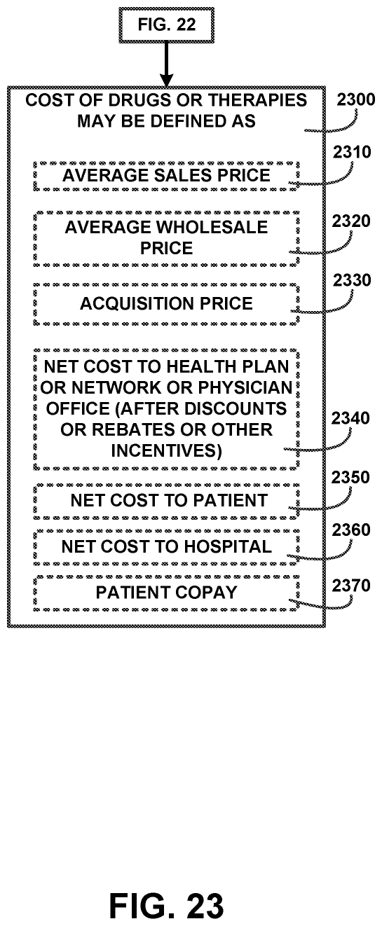

The process to evaluate cost of most effective therapies 2250 is significant as health plan or hospital or network considers using least expensive of the most effective therapies 2260, physician or practice considers using least expensive of the most effective therapies 2262, patient considers using the least expensive of the most effective therapies 2264, and state or federal government or governmental agency considers using the least expensive of the most effective therapies 2266 of one embodiment. Additional descriptions continue in FIG. 23.

### Cost of Drugs or Therapies Defined:

FIG. 23 shows a block diagram of an overview of cost of drugs or therapies defined of one embodiment. FIG. 23 shows continuing from FIG. 22 that cost of drugs or therapies may be defined as 2300, average sales price 2310, average wholesale price 2320, acquisition price 2330, net cost to health plan or network or physician office (after discounts or rebates or other incentives) 2340, net cost to patient 2350, net cost to hospital 2360, and patient copay 2370 of one embodiment.

15.0 a Method to Promote Immune Therapy Effects of Immuno-Active Drugs and/or Immune Cells in Treating Cancer or Leukemia:

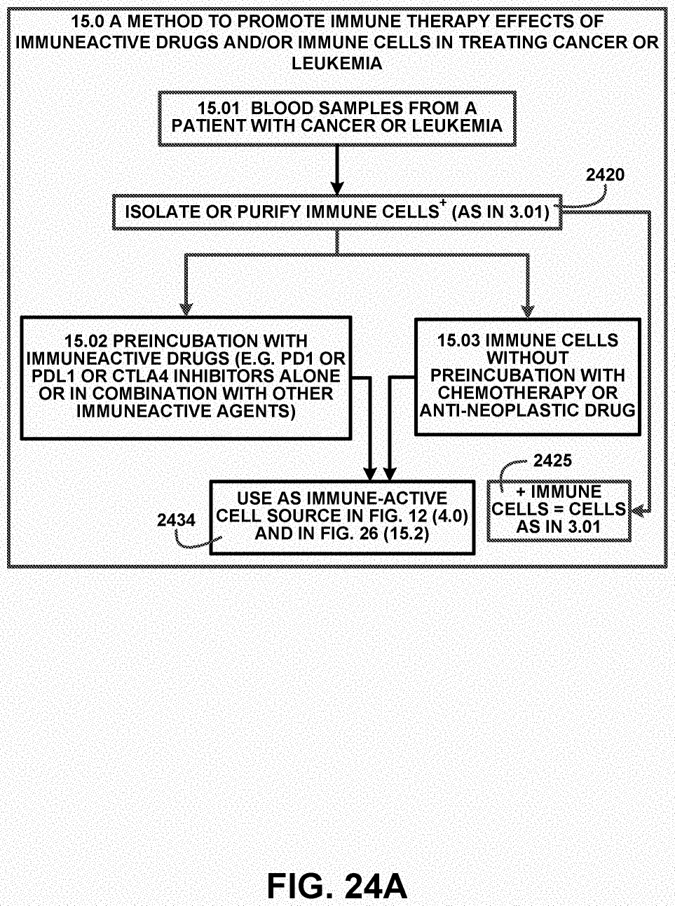

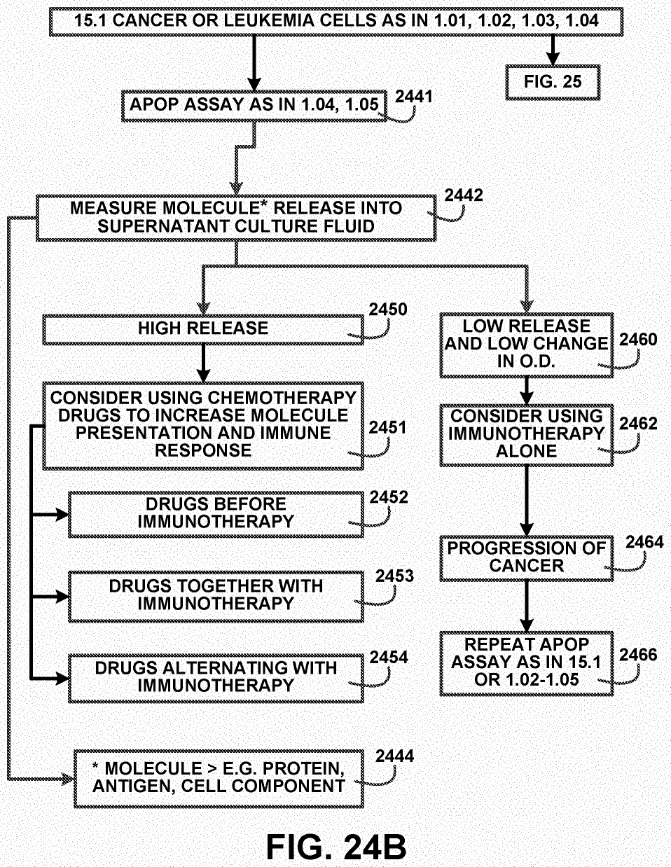

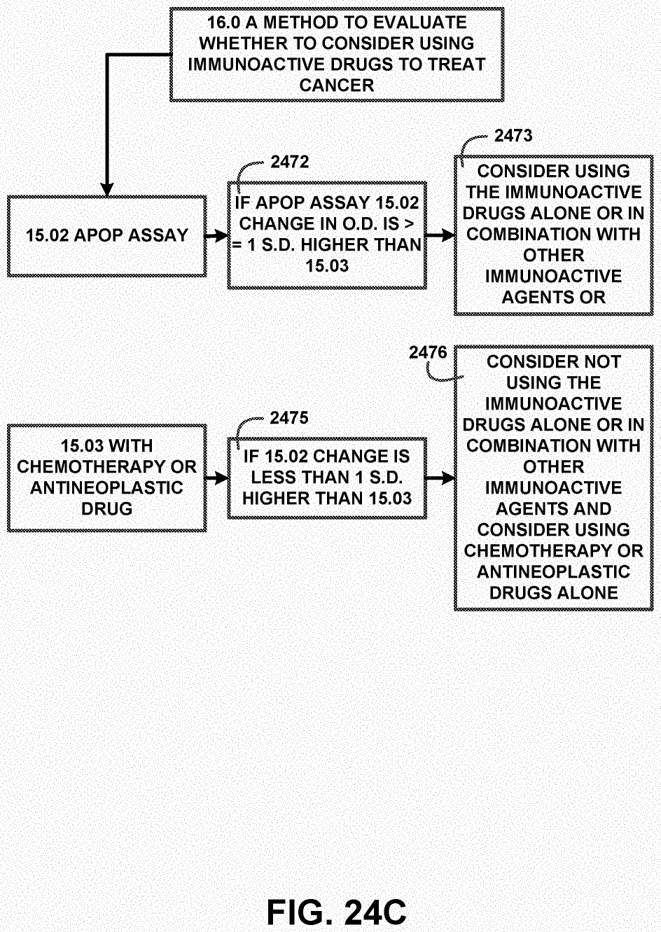

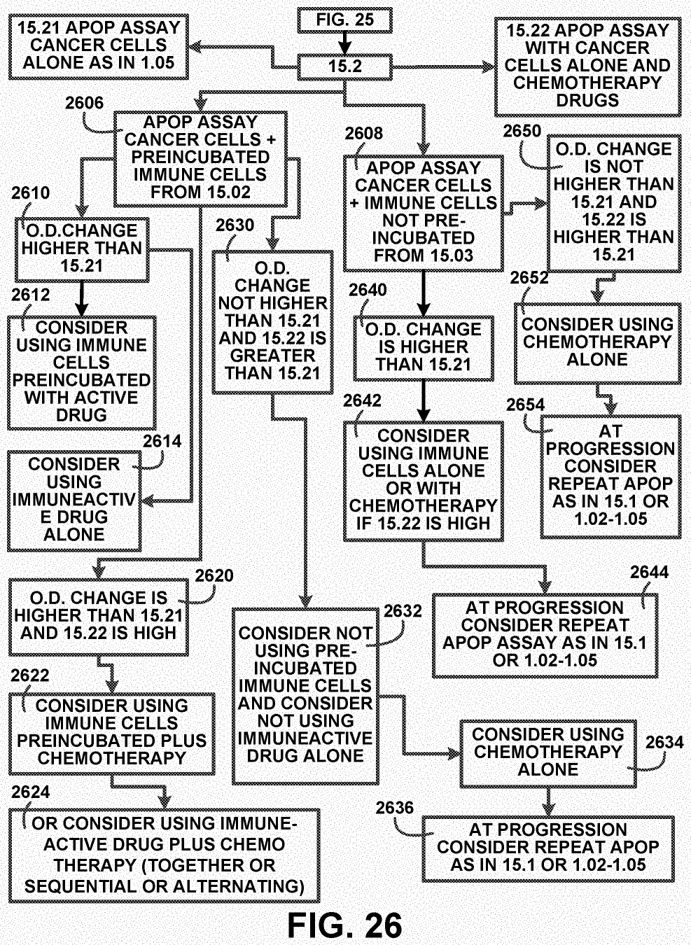

FIG. 24A shows a block diagram of an overview of 15.0 a method to promote immune therapy effects of immuno-active drugs and/or immune cells in treating cancer or leukemia of one embodiment. FIG. 24A shows step 15.0 a method to promote immune therapy effects of immuno-active drugs and/or immune cells in treating cancer or leukemia 2400. The process includes 15.01 blood samples from a patient with cancer or leukemia 2410. A process to isolate or purify immune cells⁺ (as in 3.01) 2420 where ⁺ immune cells=cells as in 3.01 2425. Processing continues with 15.02 preincubation with immuno-active drugs (e.g. PD1 or PDL1 or CTLA4 inhibitors alone or in combination with other immuno-active agents) 2430 and use as immune-active cell source in FIG. 12 (4.0) and in FIG. 26 (15.2) 2434. Including a process for 15.03 immune cells without preincubation with chemotherapy or antineoplastic drug 2432 and use as immuno-active cell source in FIG. 12 (4.0) and in FIG. 26 (15.2) 2434 of one embodiment.

### 15.1 Cancer or Leukemia Cells:

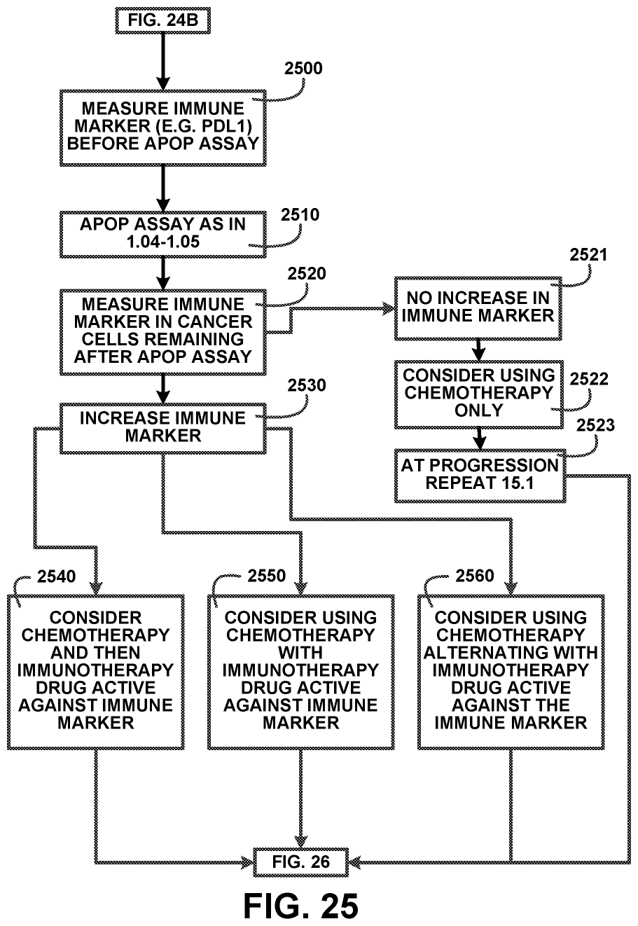

FIG. 24B shows a block diagram of an overview of 15.1 cancer or leukemia cells of one embodiment. FIG. 24B shows step 15.1 cancer or leukemia cells as in 1.01, 1.02, 1.03, 1.04 2440. The process with 15.1 cancer or leukemia cells as in 1.01, 1.02, 1.03, 1.04 2440 further continues in FIG. 25. The process with 15.1 cancer or leukemia cells as in 1.01, 1.02, 1.03, 1.04 2440 includes an APOP assay as in 1.04, 1.05 2441. The APOP assay as in 1.04, 1.05 2441 includes a process to measure molecule* release into supernatant culture fluid 2442 where * molecule >e.g. protein, antigen, cell component 2444. A high release 2450 prompts to consider using chemotherapy drugs to increase molecule presentation and immune response 2451 including drugs before immunotherapy 2452, drugs together with immunotherapy 2453, and drugs alternating with immunotherapy 2454. A low release and low change in O.D. 2460 prompts to consider using immunotherapy alone 2462 wherein a progression of cancer 2464 leads to repeat APOP assay as in 15.1 or 1.02-1.05 2466 of one embodiment.

### 16.0 a Method to Evaluate Whether to Consider Using Immunoactive Drugs to Treat Cancer:

FIG. 24C shows a block diagram of an overview of 16.0 a method to evaluate whether to consider using immunoactive drugs to treat cancer of one embodiment. FIG. 24C shows a method to evaluate whether to consider using immunoactive drugs to treat cancer 2470. The method to evaluate whether to consider using immunoactive drugs to treat cancer 2470 includes step 15.02 APOP assay 2471 and if APOP assay 15.02 change in O.D. is >=1 S.D. higher than 15.03 2472 then consider using the immunoactive drugs alone or in combination with other immunoactive agents or 2473 of one embodiment.

The 15.02 APOP assay 2471 is also performed in in step 15.03 with chemotherapy or antineoplastic drug 2474, if 15.02 change is less than 1 S.D. higher than 15.03 2475 then consider not using the immunoactive drugs alone or in combination with other immunoactive agents and consider using chemotherapy or antineoplastic drugs alone 2476 of one embodiment.

### Measure Immune Marker Before APOP Assay:

FIG. 25 shows a block diagram of an overview of measure immune marker before APOP assay of one embodiment. FIG. 25 shows a continuation from FIG. 24B from step 15.1 cancer or leukemia cells as in 1.01, 1.02, 1.03, 1.04 2440 of FIG. 24B with a process to measure immune marker (e.g. PDL1) before APOP assay 2500. The process includes performing an APOP assay as in 1.04-1.05 2510. A process in the APOP assay as in 1.04-1.05 2510 will measure immune marker in cancer cells remaining after APOP assay 2520.

If no increase in immune marker 2521 then consider using chemotherapy only 2522 and at progression repeat 15.1 2523. This process continues in FIG. 26 of one embodiment.

If there is an increase immune marker 2530 consider chemotherapy and then immunotherapy drug active against immune marker 2540 then proceed to the processes in FIG. 26 of one embodiment.

If there is an increase in immune markers 2530 consider using chemotherapy with immunotherapy drug active against immune marker 2550 then proceed to the processes in FIG. 26 of one embodiment.

If there is an increase immune marker 2530 consider using chemotherapy alternating with immunotherapy drug active against the immune marker 2560 then proceed to the processes in FIG. 26 of one embodiment. If drugs are alleged before testing to be biosimilar or identical but testing with APOP or other tests are found not to be equivalent, then neither drug may be sold as biosimilar or equivalent; this may help extend marketing of the original drug and force a putative biosimilar to undergo further testing and not be marketed.

### 15.2 APOP Assay Cancer Cells:

FIG. 26 shows a block diagram of an overview of 15.2 APOP assay cancer cells of one embodiment. FIG. 26 shows a continuation from FIG. 25 with step 15.2 2600. Step 15.2 2600 includes 15.21 APOP assay cancer cells alone as in 1.05 2602 and 15.22 APOP assay with cancer cells alone and chemotherapy drugs 2604. Step 15.2 2600 also includes an APOP assay cancer cells+preincubated immune cells from 15.02 2606 where with O.D. change higher than 15.21 2610 consider using immune cells preincubated with active drug 2612 and consider using immuno-active drug alone 2614 of one embodiment.

An APOP assay cancer cells+preincubated immune cells from 15.02 2606 where an O.D. change is higher than 15.21 and 15.22 is high 2620 consider using immune cells preincubated plus chemotherapy 2622 or consider using immuno-active drug plus chemo therapy (together or sequential or alternating) 2624 of one embodiment.

An APOP assay cancer cells+preincubated immune cells from 15.02 2606 with an O.D. change not higher than 15.21 and 15.22 is greater than 15.21 2630 consider not using pre-incubated immune cells and consider not using immune-active drug alone 2632 and consider using chemotherapy alone 2634 and at progression consider repeat APOP as in 15.1 or 1.02-1.05 2636 of one embodiment.

An APOP assay cancer cells+immune cells not pre-incubated from 15.03 2608 where an O.D. change is higher than 15.21 2640 consider using immune cells alone or with chemotherapy if 15.22 is high 2642 and at progression consider repeat APOP assay as in 15.1 or 1.02-1.05 2644 of one embodiment.

In the APOP assay cancer cells+immune cells not pre-incubated from 15.03 2608 where an O.D. change is not higher than 15.21 and 15.22 is higher than 15.21 2650 consider using chemotherapy alone 2652 and at progression consider repeat APOP as in 15.1 or 1.02-1.05 2654 of one embodiment.

### 17.0 a Method to Identify Non-Equivalences of Drugs:

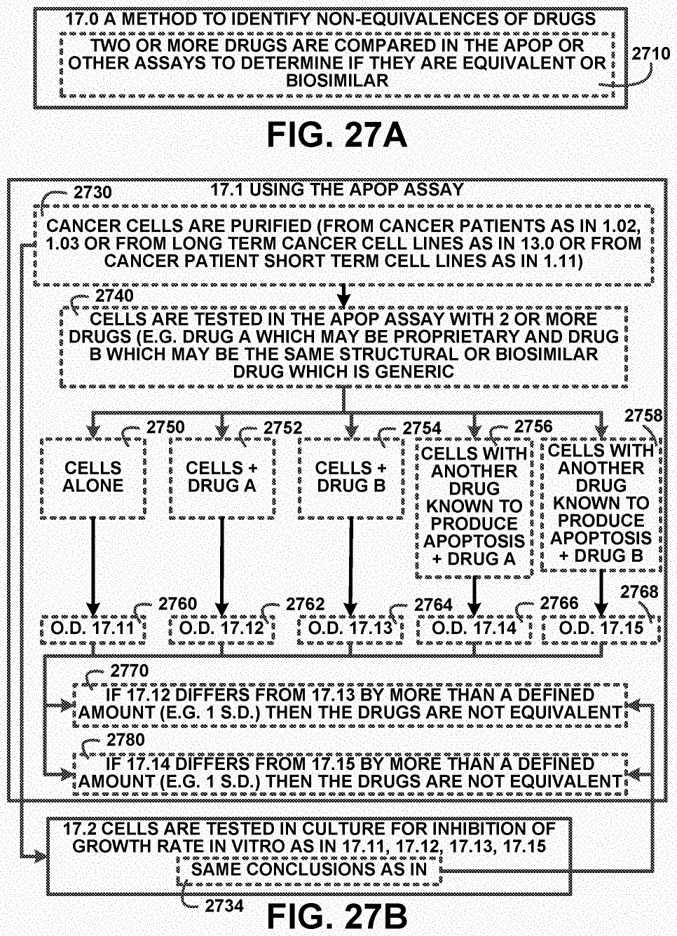

FIG. 27A shows a block diagram of an overview of 17.0 a method to identify non-equivalences of drugs of one embodiment. FIG. 27A shows step 17.0 a method to identify non-equivalences of drugs 2700. Step 17.0 a method to identify non-equivalences of drugs 2700 is a process where two or more drugs are compared in the APOP or other assays to determine if they are equivalent or biosimilar 2710. If drugs are alleged before testing to be biosimilar or identical but testing with APOP or other tests are found not to be equivalent, then neither drug may be sold as biosimilar or equivalent; this may help extend marketing of the original drug and force a putative biosimilar to undergo further testing and not be marketed. This may identify other comparable drugs that may have equal or greater effectiveness and may be able to reduce cost with their use of one embodiment.

### 17.1 Using the APOP Assay:

FIG. 27B shows a block diagram of an overview of 17.1 using the APOP assay of one embodiment. FIG. 27B shows step 17.1 using the APOP assay 2720 where cancer cells are purified (from cancer patients as in 1.02, 1.03 or from long term cancer cell lines as in 13.0 or from cancer patient short term cell lines as in 1.11) 2730. Cells are tested in the APOP assay with 2 or more drugs (e.g. drug A which may be proprietary and drug B which may be the same structural or biosimilar drug which is generic 2740 of one embodiment.

The testing includes cells alone 2750 with O.D. 17.11 2760; cells+drug A 2752 with O.D. 17.12 2762; cells+drug B 2754 with O.D. 17.13 2764; cells with another drug known to produce Apoptosis+drug A 2756 with O.D. 17.14 2766; and cells with another drug known to produce Apoptosis+drug B 2758 with O.D. 17.15 2768. If 17.12 differs from 17.13 by more than a defined amount (e.g. 1 S.D.) then the drugs are not equivalent 2770. If 17.14 differs from 17.15 by more than a defined amount (e.g. 1 S.D.) then the drugs are not equivalent 2780 of one embodiment.

Cancer cells are purified (from cancer patients as in 1.02, 1.03 or from long term cancer cell lines as in 13.0 or from cancer patient short term cell lines as in 1.11) 2730 then 17.2 cells are tested in culture for inhibition of growth rate in vitro as in 17.11, 17.12, 17.13, 17.15 2732. Testing results reach same conclusions as in 2734, if 17.12 differs from 17.13 by more than a defined amount (e.g. 1 S.D.) then the drugs are not equivalent 2770 and if 17.14 differs from 17.15 by more than a defined amount (e.g. 1 S.D.) then the drugs are not equivalent 2780 of one embodiment.

### 18.0 a Method for Identifying an Anti-Apoptosis Drug:

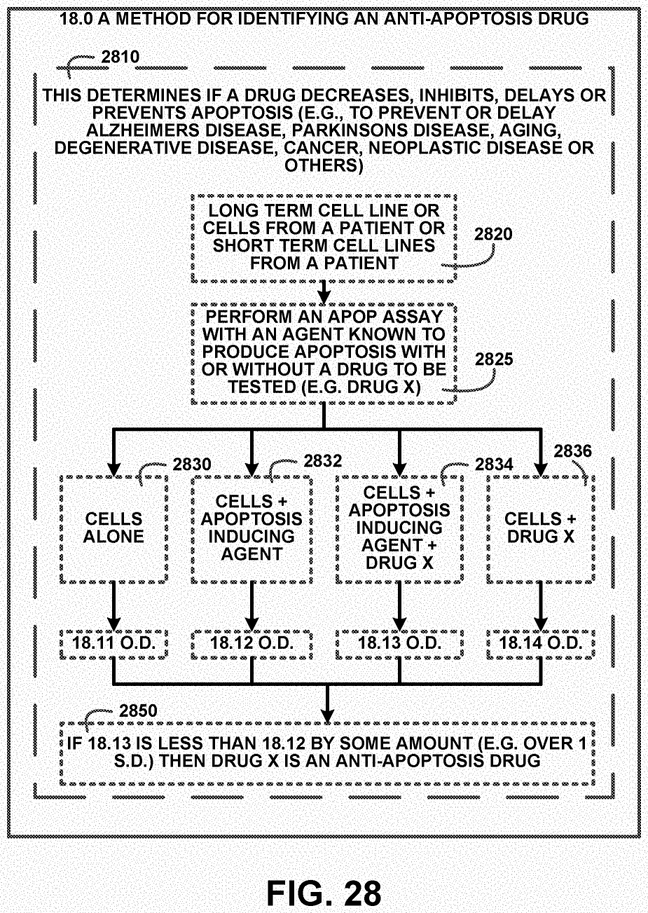

FIG. 28 shows a block diagram of an overview of 18.0 a method for identifying an anti-Apoptosis drug of one embodiment. FIG. 28 shows step 18.0 a method for identifying an anti-Apoptosis drug 2800. This determines if a drug decreases, inhibits, delays or prevents Apoptosis (e.g., to prevent or delay Alzheimer's disease, Parkinson's disease, aging, degenerative disease, cancer, Neoplastic disease or others) 2810.

The 18.0 a method for identifying an anti-Apoptosis drug 2800 uses long term cell line or cells from a patient or short term cell lines from a patient 2820 and perform an APOP assay with an agent known to produce Apoptosis with or without a drug to be tested (e.g. drug X) 2825. The APOP assay with an agent known to produce Apoptosis with or without a drug to be tested (e.g. drug X) 2825 includes cells alone 2830 with 18.11 O.D. 2840; cells+Apoptosis inducing agent 2832 with 18.12 O.D. 2842; cells+Apoptosis inducing agent+drug X 2834 with 18.13 O.D. 2844; and cells+drug X 2836 with 18.14 O.D. 2846. If 18.13 is less than 18.12 by some amount (e.g. over 1 S.D.) then drug X is an anti-Apoptosis drug 2850 of one embodiment.

### Direct APOP Assay of Purified Cells Application:

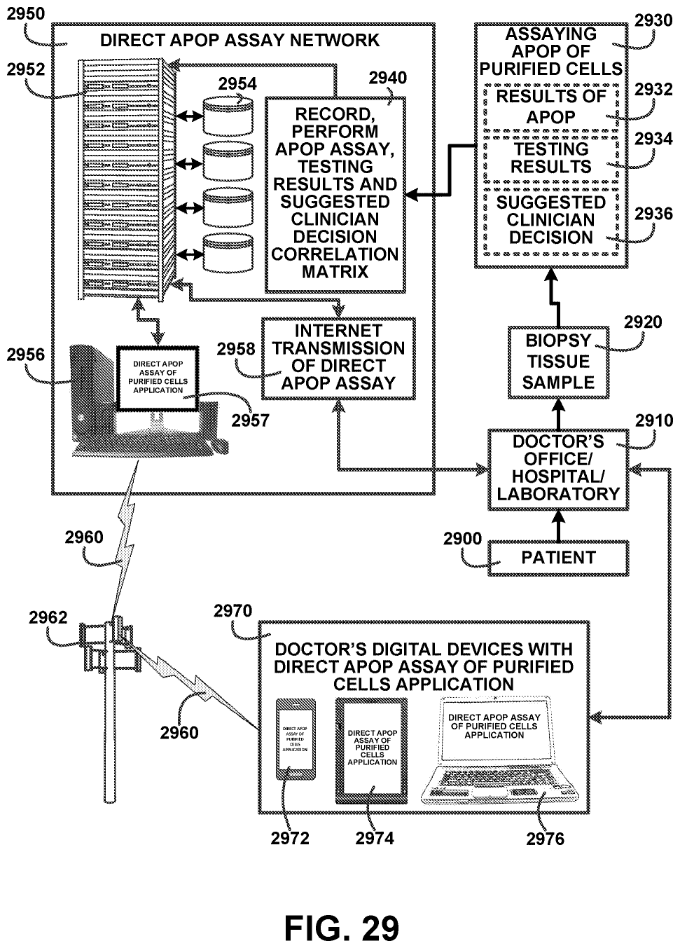

FIG. 29 shows for illustrative purposes only an example of direct APOP assay of purified cells application of one embodiment. FIG. 29 shows a direct APOP assay of purified cells application 2957 used in processing direct APOP assay results. A patient 2900 visits a doctor's office/hospital/laboratory 2910 to provide a biopsy tissue sample for determination of a diagnosis and treatment plan 2920. The patient's biopsy tissue sample 2920 is conveyed for assaying APOP of purified cells 2930. Results of APOP 2932, testing results 2934 and suggested clinician decision 2936 are transmitted to a direct APOP assay network 2950 to record, perform APOP assay, testing results and suggested clinician decision correlation matrix 2940.

The direct APOP assay network 2950 is used for controlling at least one cell purification device for purifying tissue sample cells and for example long term cancer cell lines. The direct APOP assay network 2950 is used for controlling at least one next-generation sequencer device used in performing direct APOP assay of purified cells testing. Receiving and processing tissue samples, processing using at least one cell purification device and testing using at least one next-generation sequencer device or not includes using at least one sterile enclosure of one embodiment.

The direct APOP assay network 2950 includes a plurality of digital servers 2952, a plurality of digital databases 2954, at least one computer 2956, at least one digital processor, at least one communication device with internet connectivity (not shown) 2958, at least one communication device with cellular connectivity (not shown) and at least one printer. The at least one digital processor correlates the APOP assay, testing results and suggested clinician decision data into a predetermined format including a matrix. Predetermined formats include electronic and digital formats for transmission to doctor's office/hospital/laboratory 2910 using different operating systems and computing languages and display formats. The direct APOP assay of purified cells application 2957 is configured in one embodiment to transmit the predetermined formats using internet transmission of direct APOP assay 2958 to doctor's office/hospital/laboratory 2910 computers. In another embodiment the direct APOP assay of purified cells application 2957 is configured for communicating and transmitting over cellular smart phone communication 2960 with a cellular tower 2962 to doctor's digital devices with direct APOP assay of purified cells application 2970. Doctor's digital devices including a smart cell phone 2972, a digital tablet 2974 and a laptop computer 2976 may each have a different operating system. The direct APOP assay of purified cells application 2957 is configured to operate with various operating systems of one embodiment.

The foregoing has described the principles, embodiments and modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

1-20. (canceled)

21. A drug treatment assay system, comprising:
a purification device configured for detecting and isolating live cancer cells from dead cancer cells and non-cancer cells of a patient biopsy sample;
a testing device coupled to the purification device configured to infuse at least one drug treatment to the live cancer cells;
an optical density device coupled to the testing device configured for measuring a rate of death of the live cancer cells at different intervals for a predetermined period of time after infusion of the at least one drug treatment;
a processor coupled to the optical density device configured for analyzing measured rates of death of the live cancer cells; and
at least one computer coupled to the processor configured for using analyzed rate of death results, specific patient genetic markers associated with cancer and drug resistance and allergies to generate and transmit clinician treatment recommendations for the patient's treatment based on the analyzed rate of death results of the live cancer cells testing.

22. The drug treatment assay system of claim 21, wherein the clinician treatment recommendations are used to assess anti-inflammatory therapies.

23. The drug treatment assay system of claim 21, wherein the clinician treatment recommendations are used to assess anti-immunological therapies.

24. The drug treatment assay system of claim 21, wherein the rate of death of the live cancer cells testing includes a spectrophotometric analysis at different intervals configured to collect optical density readings and/or cell count numbers for total testing times.

25. The drug treatment assay system of claim 21, wherein the processor analyzing the measured rates of death of the live cancer cells is configured for analyzing patient genomic testing for detecting genetic markers associated with cancer, drug resistance and allergy and in parallel assess where DNA mutations exists including in a tumor and bloodline mutations.

26. The drug treatment assay system of claim 21, wherein the testing device is configured to infuse at least one drug treatment including a single drug or a combination of drugs.

27. The drug treatment assay system of claim 21, wherein the optical density device is configured for measuring an increase of immune antigen stimulation to kill cancer cells and release of antigens to a patient's immune system.

28. An apparatus, comprising:
a purification isolation device configured for isolating live cancer cells of a patient biopsy sample from non-cancer cells and dead cancer cells;
a testing device coupled to the purification isolation device configured to infuse a drug or plurality of drugs to the live cancer cells;
a digital processor coupled to the testing device configured to analyze testing results with patient genomic testing for detecting genetic markers associated with cancer, drug resistance and allergies;
an optical density device coupled to the testing device configured for measuring rates of death of the live cancer cells;
at least one correlation module coupled to the optical density device configured to correlate the rate of death of the live cancer cells testing with each drug infusion results;
a digital processor coupled to the testing device configured to analyze testing results with patient genomic testing for detecting genetic markers associated with cancer, drug resistance and allergies; and
at least one computer coupled to the at least one correlation module configured for generating clinician treatment recommendations to treat a patient with the drug infusion with a highest rate of death of the live cancer cells and transmitting the clinician treatment recommendations to the clinician for consideration for the patient's treatment.

29. The apparatus of claim 28, further comprising a network coupled with the at least one computer configured to automatically collect prior drug treatment outcomes data from patients and clinicians for analyzing effects on testing results.

30. The apparatus of claim 28, wherein the optical density device is configured to automatically repeat an optical density analysis of the rate of death of the live cancer cells at different intervals for a predetermined time.

31. The apparatus of claim 28, wherein a group of a single drug and a combination of drugs includes chemotherapy drugs, legal cannabinoids/CBD drugs, and immunotherapy drugs.

32. The apparatus of claim 28, wherein at least one correlation module is configured for identifying cannabinoid/CBD anti-tumor effects, immune-activity effects and enhancement of other drug anti-tumor effects.

33. The apparatus of claim 28, further comprising an optical microplate spectrophotometric reader coupled to the optical density device configured for measuring an increase of immune antigen stimulation treatment to kill live cancer cells and release of antigens to a patient's immune system.

34. A drug treatment evaluation method, comprising:
detecting and isolating live cancer cells from dead cancer cells and non-cancer cells of a biopsy sample of a patient;
infusing at least one drug treatment to the live cancer cells;
measuring a rate of death of the live cancer cells at different intervals for a predetermined period of time after infusing the at least one drug treatment;
analyzing the measured rate of death of the live cancer cells; and
using the analyzed rate of death to generate clinician treatment recommendations for the patient's treatment based on the analyzed rate of death of the live cancer cells results.

35. The drug treatment evaluation method of claim 34, wherein measuring the rate of death of the live cancer cells includes collecting optical density readings and cell count numbers for total testing times.

36. The drug treatment evaluation method of claim 34, further comprising analyzing patient genomic testing for detecting genetic markers associated with cancer, drug resistance and allergy.

37. The drug treatment evaluation method of claim 34, further comprising collecting prior drug treatment outcomes data from patients and clinicians.

38. The drug treatment evaluation method of claim 34, wherein measuring the rate of death of the live cancer cells includes analyzing patient genomic testing for detecting genetic markers associated with cancer, drug resistance and allergies to assess the measured rate of death of the live cancer cells of drug treatments specific to that patient's current condition including genetics and prior treatment affects.

39. The drug treatment evaluation method of claim 34, wherein measuring a rate of death of the live cancer cells includes collecting optical density readings and cell count numbers for total testing times.

40. The drug treatment evaluation method of claim 34, wherein measuring the rate of death of the live cancer cells includes measuring a patient's at least one drug treated live cancer cells molecule release into a supernatant culture fluid including at least one from a group consisting of a protein, antigen, and cell component molecule.
